(19) United States
(12) Patent Application Publication
(10) Pub. No.: US 2025/0154448 A1
(43) Pub. Date: May 15, 2025

(54) DEVICE AND METHOD FOR MANIPULATING BIOLOGICAL CELLS AND METHOD OF MANUFACTURING THE DEVICE

(71) Applicants: Fraunhofer-Gesellschaft zur Foerderung der Angewandten Forschung E.V., Munich (DE); Universitaet des Saarlandes, Saarbruecken (DE)

(72) Inventors: Heiko ZIMMERMANN, Berlin (DE); Günter R. FUHR, Berlin (DE)

(21) Appl. No.: 18/840,387

(22) PCT Filed: Feb. 23, 2022
(86) PCT No.: PCT/EP2022/054515

§ 371 Date: Aug. 21, 2024

(51) Int. Cl. C12M 1/00; C12M 1/32

(52) U.S. Cl. CPC C12M 47/04; C12M 23/12; C12M 23/20

(57) ABSTRACT

The invention relates to a device (10) for manipulating biological cells, the device (10) comprising: at least one container (22) for cultivating biological cells, the container (22) having an interior space (11), and at least one electrode (14) for manipulating the biological cells, wherein the device (10) comprises at least one separator layer (20), the at least one separator layer (20) being arranged at least between the at least one electrode (14) and the interior space (11) such that the at least one electrode (14) is arranged outside of the interior space (11). The invention further relates to a method (300) for manipulating biological cells. The invention provides a device (10) and a method (300) for manipulating biological cells that avoids the degradation of the cells in containers (22) during the manipulation of the cells with electric fields.

The present invention relates to a device and a method for manipulating biological cells and a method of manufacturing the device.

For the generation of organoids and surface tissue systems, biological cells must be placed in defined neighborhoods, grouped, suitably maintained in a stable formation, or brought into defined contact with a surface in a reproducible and highly parallel manner. In automated systems or lines, microwell plates of the formats 96, 384 and 1536 wells with well-volumes of 100 μl to 300 μl, 30 μl to 100 μl, and 5 μl to 15 μl, respectively, may be used for a multiplication culture of biological cells in a stochastic arrangement.

The cell densities in biotechnological and medical applications widely vary in a range from 105 cells/ml which is 102 cells/pl. Thus, the number of cells may range from several tens of thousands in a well of a 96 microwell plate to up to thousand cells in a well of a 1536 microwell plate. Those cells are stochastically distributed in the nutrient solution in the well and may sediment on the bottom of the well.

From G. Fuhr and T. Schnelle, Dielektrische Mikrofeldkäfige, Physikalische Blätter 57 (2001) Nr. 1, pages 49 to 52, it is known to generate a defined neighborhood for those cells or an aggregation of those cells with micro electrode systems in the wells of the microwell plates. The micro electrode systems may generate electromagnetic field cages to manipulate the biological cells in the wells in a contactless manner. The cells can then be formed to aggregates. Furthermore, the cells can be positioned with micrometer precision and/or levitated in the nutrient solution.

However, electrode processes which occur close to the microelectrodes result in metal ions from the electrodes entering the solution, alkalization and acidification regions in the solution and temperature gradients in the range of 3° C. to 10° C., highest near the electrode surfaces and decreasing toward the field minimum between the electrodes. Due to those processes, mammalian or human cells degrade after about 30 minutes of continuous use of the electric fields.

Thus, there is a need to provide a device and a method for manipulating biological cells and a method of manufacturing the device that avoids the degradation of the cells in the wells during the manipulation of the cells with electric fields.

The object of the present invention is solved by the subject-matter of the independent claims. Further embodiments are incorporated in the dependent claims.

According to the present invention, a device for manipulating biological cells is provided, the device comprising: at least one container for cultivating biological cells, the container having an interior space, and at least one electrode for manipulating the biological cells, wherein the device comprises at least one separator layer, the at least one separator layer being arranged at least between the at least one electrode and the interior space such that the at least one electrode is arranged outside of the interior space.

The invention provides a separator layer between the at least one electrode for manipulating the biological cells and the interior space of the at least one container. The at least one electrode is assigned to the at least container, such that each container of the device comprises its own electrode. Furthermore, the at least one electrode is arranged outside the interior space of that container and does not have any direct contact to the interior space. Thus, the separator layer shields the interior space from the electrode material, such that the electrode does not have any physical contact to a suspension that may be arranged in the interior space of the at least one container, wherein the suspension comprises a nutrient solution and the cells. Therefore, the at least one electrode may also be called dry electrode. The at least one electrode capacitively couples an electric field into a liquid being arranged in the interior space. In other words the electrode couples an electric field indirectly into the liquid, i.e. without a charge transfer or flux between the electrode and the liquid. Thus, the pure displacement current in the electrode without ohmic line contribution is sufficient to provide an electric field strong enough for manipulating the biological cells in the container. In this description, the term cell refers to a biological cell. Electrode processes are avoided during this special application of the electric field. Due to a lack of degradation of the cells, the at least one electrode may apply an electric field on the cells for an unlimited period of time.

In an example, the at least one electrode may be a microelectrode. Furthermore, in an example, the at least one container may be a well of a microwell of a microwell plate.

In an example, at least one bottom element of the at least one container may comprise the separator layer and the at least one electrode may be arranged outside the interior space at the bottom element.

The bottom element of the at least one container may be attached to walls of the container and close the bottom of the container in a fluid tight manner. Furthermore, the bottom element may comprise only the separator layer such that the separator layer is the bottom element. The at least one electrode is then arranged below the bottom element. One side of the bottom element may face the interior space, wherein the opposite side of the bottom element may face the at least one electrode. This arrangement provides an effective shielding of the interior space from the electrode material.

In another example, the at least one bottom element may comprise a thickness of at most 200 μm, preferably of at most 100 μm, further preferably of at most 25 μm, at least at the at least one electrode.

The thickness of the bottom element is then reduced such that the bottom element has a reduced mitigation of an electric field of the at least one electrode.

In a further example, the at least one electrode may be arranged between the bottom element and an electrically insulating layer.

The electrically insulating layer may shield the at least one electrode from a user or from further devices below the device for manipulating biological cells. Furthermore, the insulating layer may stabilize the bottom element and the device for manipulating biological cells.

Furthermore, the at least one electrode may for example be arranged at least partially in the at least one container and, at least inside the container, is coated with the separator layer.

A section of the at least one electrode may be arranged inside the container. Furthermore, the complete surface of the electrode that is arranged inside the container may be coated with the separator layer. Thus, the separator layer separates the electrode from the interior space. The surface of the electrode does not have any direct contact to a nutrition solution nor cells that are arranged in the interior space.

In another example, the separator layer may comprise materials with a relative permittivity in a range from 10 to 10000, preferably from 10 to 1000, further preferably from 20 to 500 for voltage-current waveforms with a frequency in the range from 1 kHz to 10 MHz and preferably comprises ferromagnetic properties.

The term relative permittivity is also known as dielectric constant. The higher the relative permittivity of the separator layer, the higher the electric field in the interior space generated by the at least one electrode. Furthermore, the higher the relative permittivity, the thicker the separator may be layer without changing the strength of the electric field inside the interior space. The voltage applied on the at least one electrode to generate the electric field may also be reduced if the relative permittivity is high.

The separator layer may, for example, comprise particles having a crystal structure, preferably micro-crystals, the particles having a uniform size in the range from 100 nm to 1000 nm, preferably 300 nm, or the particles having different sizes in the range from 100 nm to 1000 nm, preferably 300 nm and 700 nm.

The particles may for example have a high relative permittivity of more than 100 up to 10000 wherein the particles are embedded in a main material of the separator layer having a relative permittivity of around 10 or higher. Thus, the particles may increase the total relative permittivity of the separator layer.

In a further example, the particles may be arranged in a columnar manner in the separator layer between two opposite sides of the separator layer.

Such an arrangement of the particles in the separator layer may further increase the relative permittivity of the separator layer if the particles have a higher relative permittivity than the main material of the separator layer.

In another example, the separator layer may comprise a titanate of an alkaline earth metal, preferably CaTio₃, SrTiO₃, BaTiO₃, Ba₁₋ₓSrₓTiO₃ and/or combinations thereof, preferably in a ratio between 10% to 60% by volume, further preferably between 30% and 50% by volume, most preferably of at most 40% by volume.

The titanate of an alkali earth metal may for example be the microcrystals mentioned above. Those titanates have a high relative permittivity. The ratio of the titanate provides a separator having a high relative permittivity wherein the separator material may stay optically transparent. Since the separator material is arranged on the bottom of the container, optical transparency allows the use of optical means for observing the cells in the container.

In an example, the separator layer may comprise at least one polymer, preferably a cyano resin, more preferably cyanoethyl pullulan (CRS), cyanoethyl poly (vinyl alcohol) (CRV) and/or cyano resin type M (CRM), further preferably with an relative permittivity above 10.

The at least one polymer simplifies the manufacturing of the separator layer. In the beginning of the manufacturing process, the polymer is liquid. Particles for increasing the total relative permittivity may be mixed with the liquid polymer. The solidification of the polymer, for example by irradiation with ultraviolet light, may fix orientation and the position of the particles in the polymer.

The separator layer may for example have a total relative permittivity in a range of from 10 to 200, preferably from 16 to 120, more preferably from 20 to 120, for voltage-current waveforms having a frequency in the range of from 1 kHz to 10 MHZ.

Then, electric field lines from the at least one electrode may pass the separator layer without significant attenuation. This simplifies the manipulation of the cells or cell groups in the container. In the context of this description, the terms cell group, cell aggregate, cell cluster and organoid are used synonymously. Furthermore, due to the high permittivity of the separator layer, the voltage required for the manipulating electric field can stay in a range being non-hazardous for a user.

In a further example, the separator layer may comprise at least one curved surface region at the interior space.

Furthermore, the at least one curved surface region may for example comprise at least one convex and/or at least one concave portion.

The surface region between the separator layer and the interior space is a material boundary between materials with different relative permittivities. Electric field lines from the at least one electrode are refracted at that material boundary. The shape/geometry of that surface region may be formed to achieve a desired electric field pattern within the container. For example, a curved surface region may shape the electric field lines in the interior space differently from a flat surface region. This may simplify and/or support the forming and/or manipulation of the cells or cell groups.

In another example, the separator layer may comprise at least two regions, each one of the at least two regions having a different relative permittivity, wherein the at least two regions preferably comprise different materials and/or different material mixture ratios.

The different regions may also shape the electric field lines in the interior space to simplify and/or support the forming and manipulation of the cells or cell groups. In combination with regions with curved surfaces, the simplification and/or support effects increase, further. In particular, cells or cell groups with different distribution patterns may be formed in that manner.

The at least one electrode may for example comprise a base metal, in particular aluminum or nickel; an alloy of base metals; or at least one plotter-writable conductive ink or paste.

Since the at least one electrode does not have any contact to the interior space, particularly to the nutrient solution of the cells, the material of the electrode does not need to have the properties of a noble metal. Furthermore, also alloys or toxic metals may be used for manufacturing the electrode. Thus, the at least one electrode can be manufactured from a more cost-effective material. The manufacturing of the electrode may be simplified when using a plotter-writable conductive ink or paste.

In a further example, the device may comprise at least two electrodes, preferably at least four electrodes in a quadrupole arrangement or at least eight electrodes in an octupole arrangement.

The electric field from those electrodes may simplify and/or support the manipulation of the cells or cell groups. The use of quadrupole or octupole arrangement allows for the use of electromagnetic field cages. Those cages may be used to group, shape, position and levitate the cells or cell groups in the container in a contactless manner, in general to form desired cell distribution patterns.

Furthermore, the electrodes may, for example, be electrically connected to different phases of a multiphase voltage source or may be electrically connected in pairs to one phase of a multiphase voltage source.

If the electrodes are connected in electrical connection in pairs to one phase of the multiphase voltage source, each pair may be connected to a different phase of the multiphase voltage source. The connection of the electrodes to different phases, pairwise or alone, simplifies the manipulation of cells or cells groups, particularly the positioning and levitation.

In another example, the at least one electrode may have at least one section, which is cross-shaped or Y-shaped.

In a further example, the at least one electrode comprises an end piece having a circular, triangular, square, or T-shaped cross-sectional area.

Furthermore, for example, the at least one electrode may be linear or zigzag-shaped and/or may have a plurality of preferably triangular projections extending along the bottom element.

The shape of the electrode and/or the end piece of the electrode influences the generated electric field in the interior space. Thus, the shape of the electrode and/or the end piece may lead to a simplification of manipulating the cells or cell groups. The shapes mentioned above may be combined in any sensible combination.

In an example, the device may comprise a plurality of containers and a plurality of electrodes, wherein at least one of the plurality of electrodes is arranged on each container, wherein the device preferably may be formed as a microwell plate.

The device may manipulate cells and/or cell groups in all containers at the same time. Thus, a plurality of different cells and/or a plurality of different conditions in the containers may be monitored.

In a further example, a first group of the plurality of electrodes may be electrically connected to a first phase of a multiphase voltage source via a first electric line and a second group of the plurality of electrodes may be electrically connected to a second phase of the multiphase voltage source via a second electric line, wherein at least one electrode from each group is disposed on each container.

Thus, the electrodes may generate the same electric field at each container. If the separator layer in each container comprises the same properties, i.e., the same surface shape, the same thickness and the same relative permittivity, etc., the electric field in the interior space of the containers will be the same.

The device further may, for example, comprise at least one energy storage device and at least one electronic circuit for generating voltages having a frequency at least in the range between 1 kHz and 10 MHz, the at least one electronic circuit electrically connecting the at least one electrode to the at least one energy storage device.

This results in alternating electric fields in the interior space. The alternating electric field allows moving the cells or celling group in the container by positive or negative dielectrophoresis. The positivity or negativity of the dielectrophoresis depends on the applied frequency of the electric field and the physiological conductivity of the cells in the interior space.

The frequencies in the range of 1 kHz and 10 MHz are also called radiofrequencies or high frequencies in the context of this description. Those high frequencies avoid the induction of thermal currents in the suspension. Furthermore, the high frequency of the alternation of the electric field reduces the stress of the biological cells resulting from the application of an electric field.

In another example, the at least one electrode may be arranged on a first module and the at least one electronic circuit may be arranged on a second module being detachable from the first module, the at least one electrode being electrically connected to the at least one electronic circuit via a detachable electrical contact that is arranged between the first module and the second module.

The arrangement of the detachable electrical contact between the at least one electrode and the at least one electronic circuit provides for a simple manufacturing of the device. Furthermore, the first module may be detached from the second module. This provides a flexible arrangement of the electronic circuit in relation to the containers.

The detachable electrical contact may comprise a gold material. Furthermore, the detachable electrical contact may comprise a spring contact.

Furthermore, the electrical contacts may comprise miniaturized digital electronic components.

For example, the device may comprise at least one conductive or insulating island element arranged electrically separated from the at least one electrode on the container, wherein the separator layer separates the conductive or insulating island element from the interior space.

The conductive islands may focus the electric field lines. Thus, when material showing positive dielectrophoresis is in the nutrient solution, that material will group at the focused electric field lines (region with a higher or highest density of electric field lines). The insulating islands may defocus the electric field lines. Thus, material showing negative dielectrophoresis will group at the defocused electric field lines (region with a lower or lowest density of electric field lines). This further simplifies the manipulation of the cells or cell groups.

In another example, the at least one conductive or insulating island element may have a length in the range of 1 μm to 200 μm.

Furthermore, in an example, the interior space may comprise at least one bead, tube and/or wire.

The cells may aggregate around the bead, tube and/or wire. Thus, if the cell aggregate grows, the bead, tube and/or wire may provide a channel for the nutrient solution into the cell aggregate. Thus, the cells in the core of the cell aggregate remains in contact with the nutrient solution. The bead, tube and/or wire may e.g. comprise natural or artificial semipermeable material.

In a further example, at least a portion of the at least one conductive of insulating island element may comprise a functional material surface. The biofunctional material surface enhances the aggregation of the cells on the islands in the nutrient solution.

The biological cells may for example be stem cells, preferably induced pluripotent stem cells.

Those kinds of cells may be used to grow tissue in the containers. The device may simplify and control the growth of tissue from those cells.

According to another aspect of the present invention, a method for manipulating biological cells by a device according the above description is provided, wherein the method comprises the following steps: introducing a suspension comprising at least one biological cell into the at least one container; applying an electrical signal to the at least one electrode to generate a variable electric field; moving the at least one biological cell to a predefined position in the container by the variable electric field, thereby manipulating the at least one biological cell.

The electrical signal applied to the at least one electrode (e.g. a voltage) results in the generation of an electric field extending through the separator layer of the device and ultimately into the interior space of the container. The variable electric field may exert a force on the at least one biological cell in the interior space by positive or negative dielectrophoresis. The force may move the at least one biological cell in the interior space. The movement may occur in every direction inside the suspension depending on the strength, direction and the frequency of the variable electric field at the position of the at least one biological cell. The cells will move towards the field maxima or minima of the electric field depending on whether positive or negative dielectrophoresis is present. Thus, by positioning the field maxima or minima, the cell may be deliberately and accurately placed at a certain position inside the interior space. Furthermore, a plurality of cells may be collected at that position to form cell groups. Since the separator layer separates the at least electrode that generates the variable electric field from the interior space, i.e., the cells and the nutrient solution, degradation of the cells is avoided.

In an example, the electrical signal may have a voltage peak-to-peak value in a range from more than 4 V to 100 V, preferably from more than 10 V to 100 V, more preferably from more than 10 V to 50 V.

In another example, the at least one electrical signal may be fed in a continuous manner or in non-continuous manner, preferably a pulsed manner. This may reduce the heat production in the electrode. Consequently, this reduces the heating of the device.

In a further example, the device may comprise at least two electrodes, wherein the electrical signal may be configured such that the at least two electrodes generate an electric field with a stable temporal pattern of minima and maxima within the container. The generation of an electric field with a stable temporal pattern may result in forces on the cells or particles in the suspension which further simplify the manipulation of the cells or cell groups.

The electric field may, for example, exert a force in the range of 1 pN to 1000 pN on a cell inside the container at a distance between 10 μm and 5 mm from the surface of proximal surface of the at least one electrode.

Furthermore, the at least one cell may, for example, be moved into at least a minimum of the electric field.

For example, the at least one cell may be polarized by the electric field.

The polarization of the at least one cell results in positive or negative dielectrophoresis such that the at least one cell is moved towards the maxima or minima of the electric field lines, respectively.

In an example, the suspension may comprise at least two cells, wherein the cells in the suspension are formed into at least one cell aggregate by the electric field.

Furthermore, the at least one cell aggregate may, for example, be sedimented on a bottom of the container by a reduction of a field strength of the electric field. Thus, the cell aggregate may be shaped when the cells are levitating. The shaped cell aggregate may be sedimented to position the cell aggregate on the bottom of the container.

In a further example, the electrical signal is switched off as soon as the at least one cell aggregate has been sedimented. In doing so, no electric field is present during the further growth of the biological cells of the cell aggregate.

In another example, a collecting force may be exerted on the at least one sedimented cell aggregate by the electric field. Thus, the electric field holds together the cells of the cell aggregate during the growth of those cells.

According to another example, the electrical signal may be applied permanently or in alternating sequence with an overall short application time of the electric field. The short application time may correspond to several hours, preferably to the time during which the cells are maintained in the well. The short time application of the electric field may have a switch-on time range from 1 ms to 1000 ms and a switch-off time range from 1 ms to 60 min.

In an example, after the step of introducing the suspension into the container, the suspension may be conditioned by at least one group of cells. This may enhance the chemistry of the suspension to optimize the condition of cell growth.

Furthermore, after conditioning the suspension, the at least one group of cells may, for example, be separated and aggregated by the electric field in the container. In this example, at least one further cell may be introduced into the suspension, wherein the at least further one cell is moved to a minimum of the electric field and held there for a predefined period. The at least one cell may be positioned in a field maximum such that the at least one cell and the at least one further cell are separated from each other. Thus allows the formation of more than one cell group in one container for co-cultivation.

In an example, the at least one cell may be observed microscopically in the container and a behavior, and an increase of the cell may be recorded.

In another example, the method may further comprise the following step before the step of introducing the suspension: generating positive or negative dielectrophoresis with respect to the at least one biological cell by selecting the suspension according to its desired conductivity and selecting a frequency of the variable electrical signal.

In an example, the at least one cell may be lifted and moved in the container by negative dielectrophoresis.

Furthermore, after the step of applying the electrical signal to the at least one electrode, the method may, for example, further comprise at least one of the following steps: cooling the at least one electrode by direct contact with a coolant while performing cryopreservation of the suspension; and/or heating the at least one electrode by feeding at least one electrical signal into the at least one electrode at a frequency in a range of 1 kHz to 10 MHZ when thawing a frozen suspension.

Since the separator layer may be formed such that it comprises a high thermal conductivity, the cooling of the suspension occurs fast. Thus, the cryopreservation of the suspension is simplified.

In another example, at least one cell may be measured electrically and/or dielectrically by an induced polarization by the electric field.

The measurement may comprise the generation of an electric field which is sufficient to induce a polarization of the at least one cell. After switching of the electric field, the at least one electrode may measure the decay of the induced polarization.

Furthermore, the biological cells may be stem cells, preferably induced pluripotent stem cells.

According to another aspect of the present invention, method of manufacturing a separator layer of the device according to the above description is provided, the method comprising the following steps: providing at least one liquid polymer having a first relative permittivity; mixing a plurality of particles, preferably microcrystals, having a second relative permittivity with the at least one liquid polymer to obtain a separator layer mixture; solidifying the separator material mixture to obtain a separator layer.

The plurality of particles and the at least one liquid polymer have different relative permittivities, wherein the polymer has a lower relative permittivity than the particles. The introduction of the particles into the polymer results in a mixture having a relative permittivity that is between the first and second permittivity. Thus, the mixture has a third relative permittivity that is higher than the first relative permittivity. The solidified separator material mixture has a smaller mitigation/attenuation of an electric field than the polymer. Overall, the method provides a device that avoids the degradation of cells during the manipulation of the cells with electric fields.

In an example, an electric field may be applied to the separator material mixture during at least a fraction of the solidification process thereof for alignment, collection, and chain formation of the particles in the liquid polymer to produce ordered, columnarly aligned particle regions and/or particle clusters in the separator layer. The ordered, columnarly aligned particle regions and/or particle clusters further increase the relative permittivity of the separator layer as compared to a homogenous mixture of the particles and the liquid polymer. The electric field may be an alternating electric field.

In another example, in the step of mixing of the plurality of particles with the at least one liquid polymer, the particles may have a uniform size and preferably a volume ratio of at most 40% with respect to the liquid polymer. The particles of uniform size result in columns having a uniform shape if an electric field is applied during the solidification of the separator layer material. The uniformly sized particles form chains along the electric field lines which result in the columns.

In a further example, in the step of mixing of the plurality of particles with the at least one liquid polymer, the particles may have at least two different sizes. When the two particle fractions form chains along the electric field lines, the particles form uniform columns. While the electric field is applied, the smaller particles move between bigger particles such that columns with heterogeneous shapes are formed. This may avoid a rigid pre-polarization of the cells.

According to an example, the separator material mixture may be solidified to form a separator layer, wherein the electric field is applied perpendicularly to separator material mixture during its solidification. Thus, the columns of the particles extend perpendicularly to the separator layer. In the device, one end section of the columns may face the electrode, wherein the opposite end section of the columns may face the interior space.

In an example, before solidifying the separator material mixture, the separator material mixture may be applied on the at least one electrode in a closed layer, preferably by sputtering, spin coating, screen printing and/or depositing in a sol-gel process.

The closed layer covers the complete surface portion of the electrode that is arranged in the container. After the layer is complete, the electrode is isolated from the interior space of the container and therefore behaves as if it was arranged outside the interior space of the container.

In a further aspect of the invention, a method of manufacturing a device according to the above description is provided, the method comprising: providing the container for cultivating biological cells, providing a layer of the separator layer, providing the at least one electrode for forming and manipulating the biological cells, wherein the electrode is separated from the interior space of the container by the separator layer.

Advantages and effects as well as further developments of the method for manufacturing the device result from the advantages and effects as well as further developments of the device and methods described above. Thus, reference is made to the preceding portion of the description.

In an example, the separator layer may be manufactured according to the method of manufacturing a separator layer of the device according to the above description.

In an even further aspect of the invention, a use of the device according to the above description for manipulating biological cells is provided.

Furthermore, in a further aspect of the invention, a use of a varying electric field coupled capacitively into and externally from a container comprising a suspension with at least one biological cell for manipulating said biological cell in the suspension is provided.

Using the device for manipulating biological cells and/or the varying electric fields opens a variety of applications which were not feasible with prior art devices. For example, since the degradation of mammal or human cells is avoided while applying the electric field, the period of the electric field application may be extended to an unlimited period. This allows to aggregate and form the cells in a manner which has not been possible before.

Exemplary embodiments of the invention will be described in the following with reference to the following drawings:

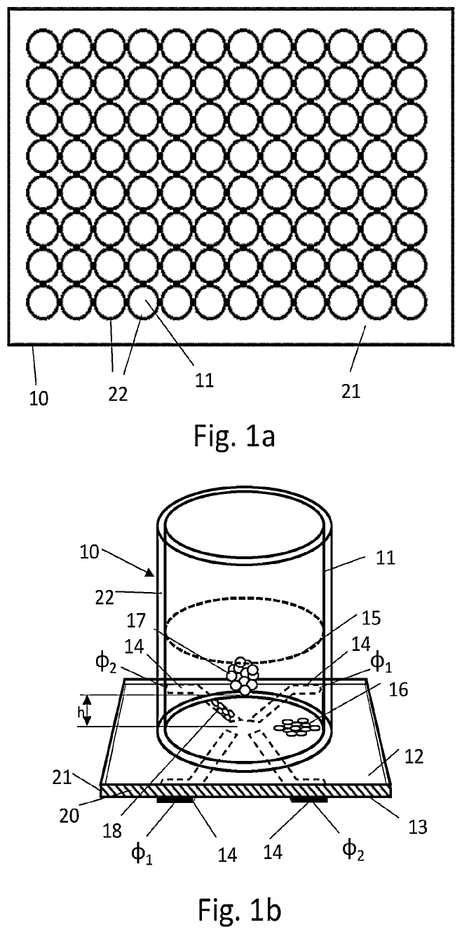

FIGS. 1a, 1b show a schematic view of a device for manipulating biological cells with containers.

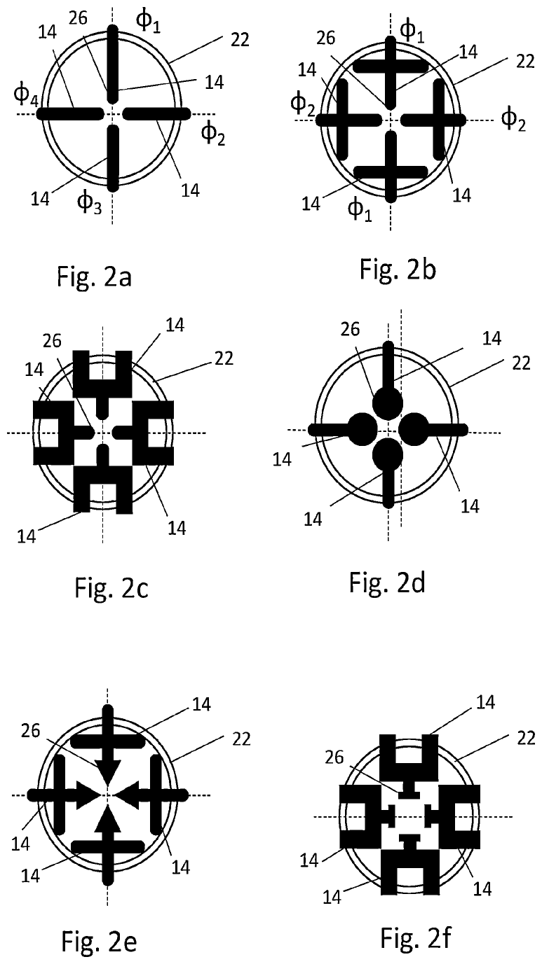

FIGS. 2a-f show a schematic view of examples of the at least one electrode.

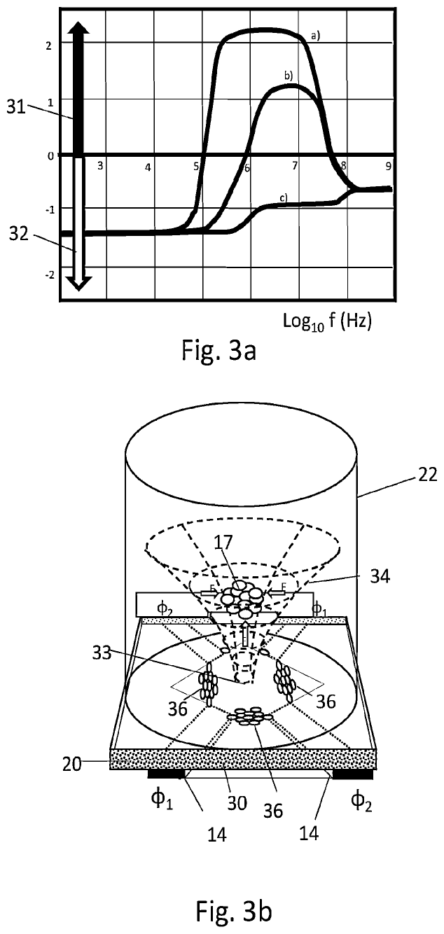

FIGS. 3a, 3b show a diagram of the electrophoresis and a container of the device.

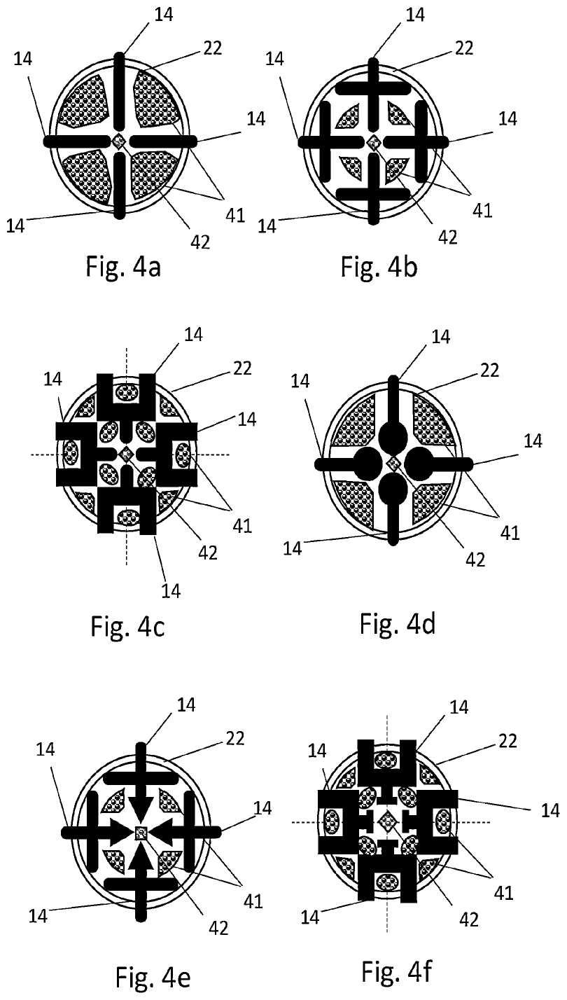

FIGS. 4a-f show a schematic view of the examples of FIGS. 2a-f with cell aggregates.

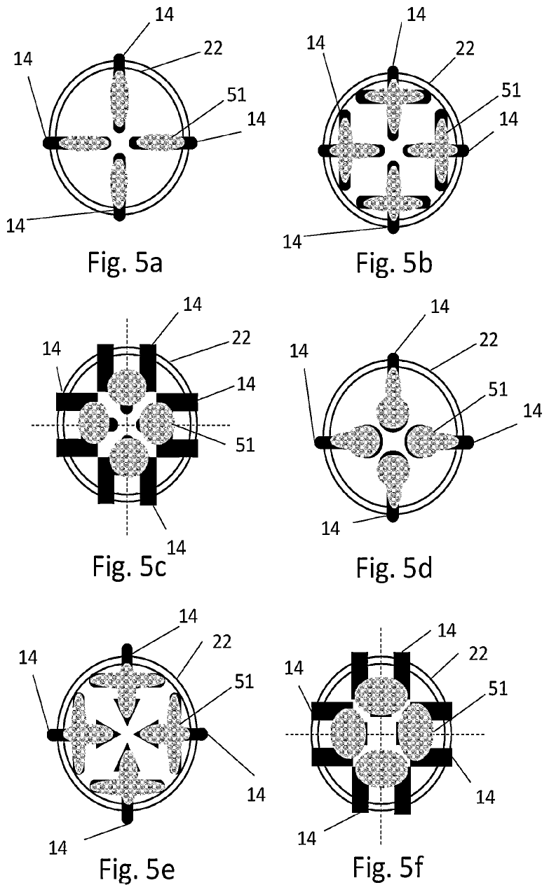

FIGS. 5a-f show a schematic view of the examples of FIGS. 2a-f with cell aggregates with positive electrophoresis.

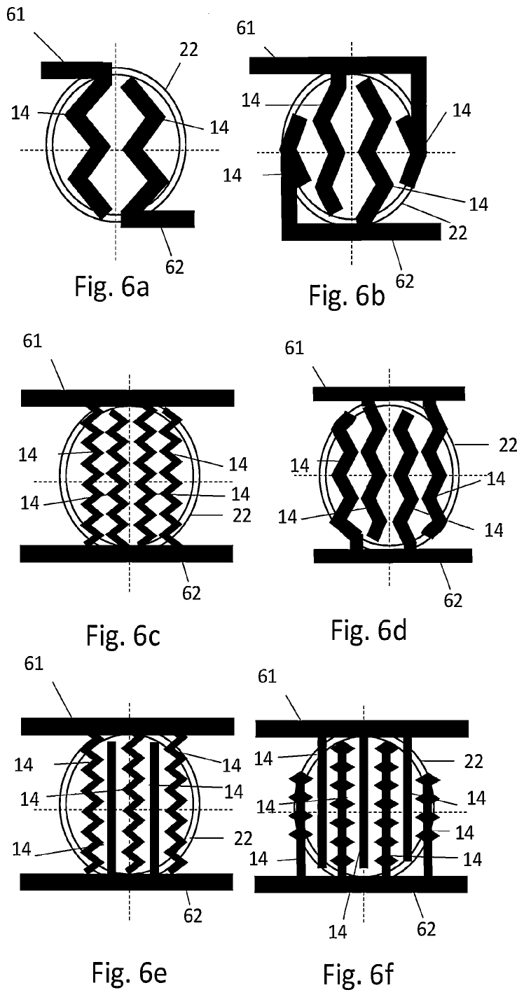

FIGS. 6a-f show a schematic view of further examples of the at least electrode.

FIG. 7 shows a schematic view of an example of a connecting electrodes of a plurality of containers.

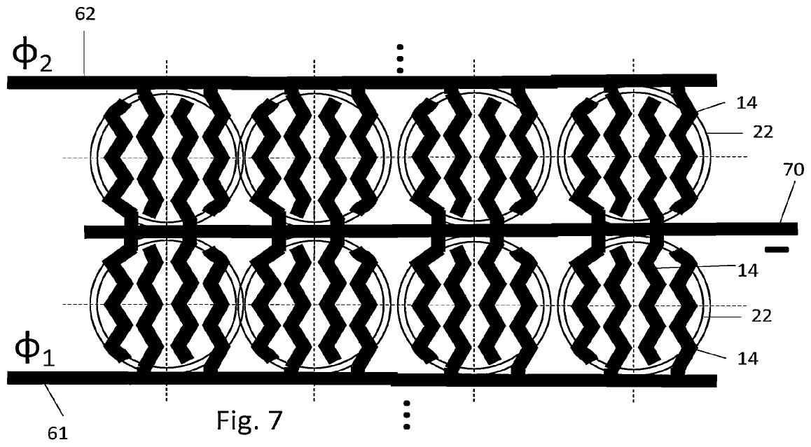

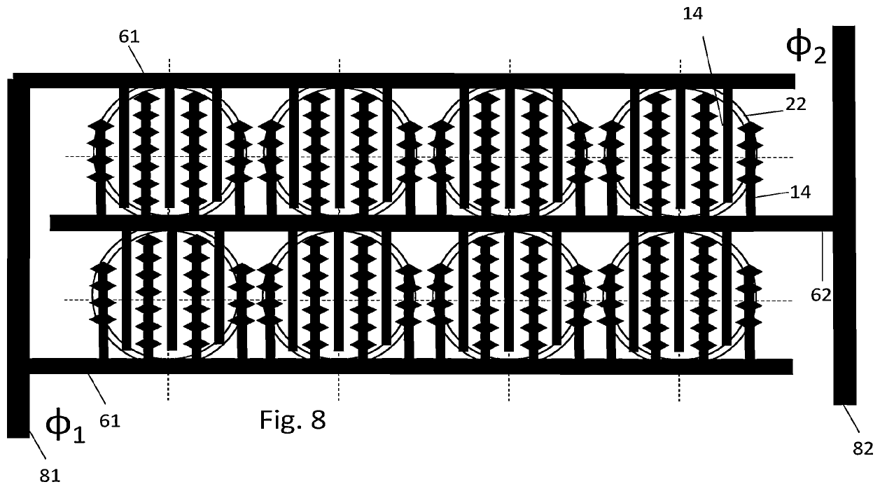

FIG. 8 shows a schematic view of another example of a connecting electrodes of a plurality of containers.

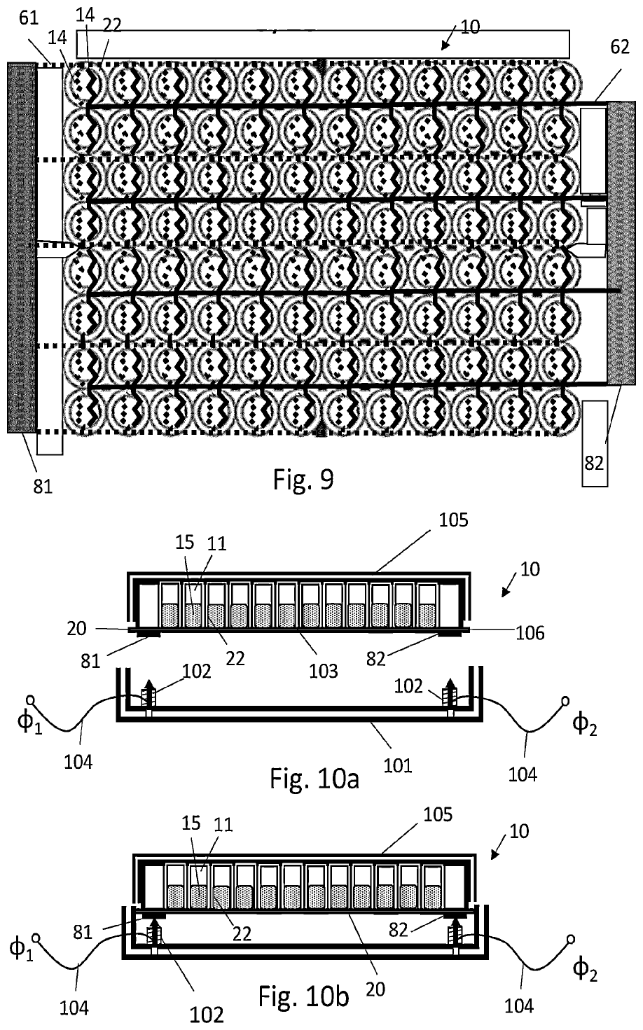

FIG. 9 shows a schematic view of an example of electrical contacts for the electrodes on the device.

FIGS. 10a, 10b show a schematic view of an example of a first and second module of the device.

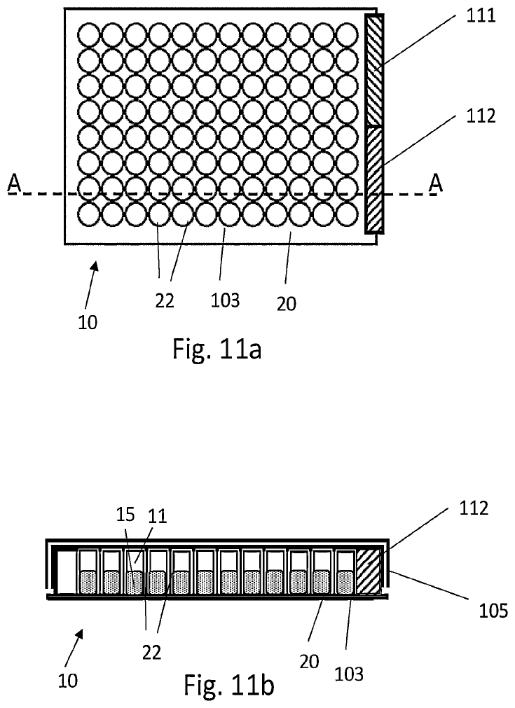

FIGS. 11a, 11b show a schematic view of an example of the device with electronic circuits.

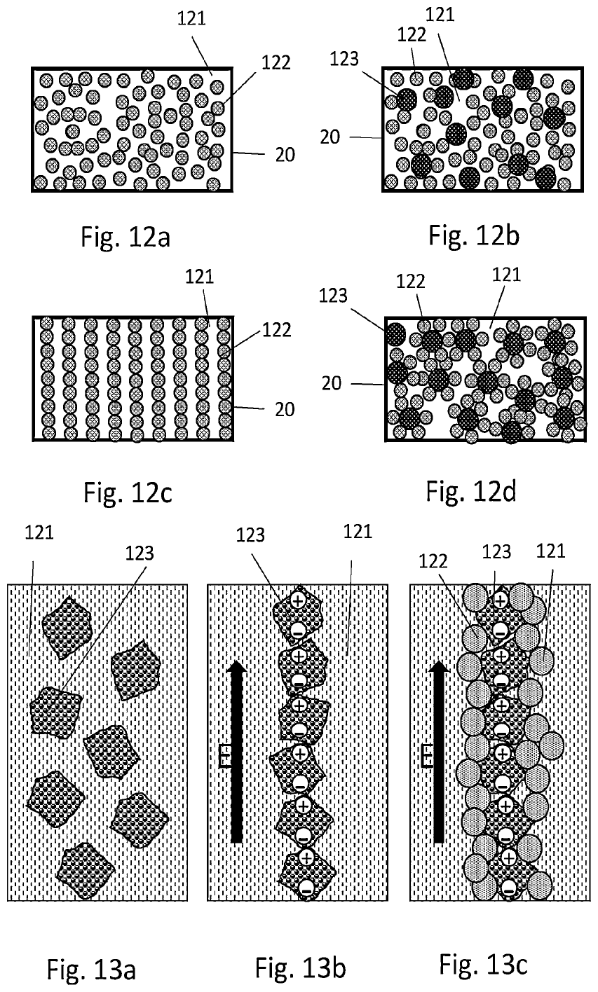

FIGS. 12a-d show a schematic view of examples of inner structure of the separator layer.

FIGS. 13a-c show a schematic view of the forming of columnar structures according to FIG. 12b and FIG. 12d.

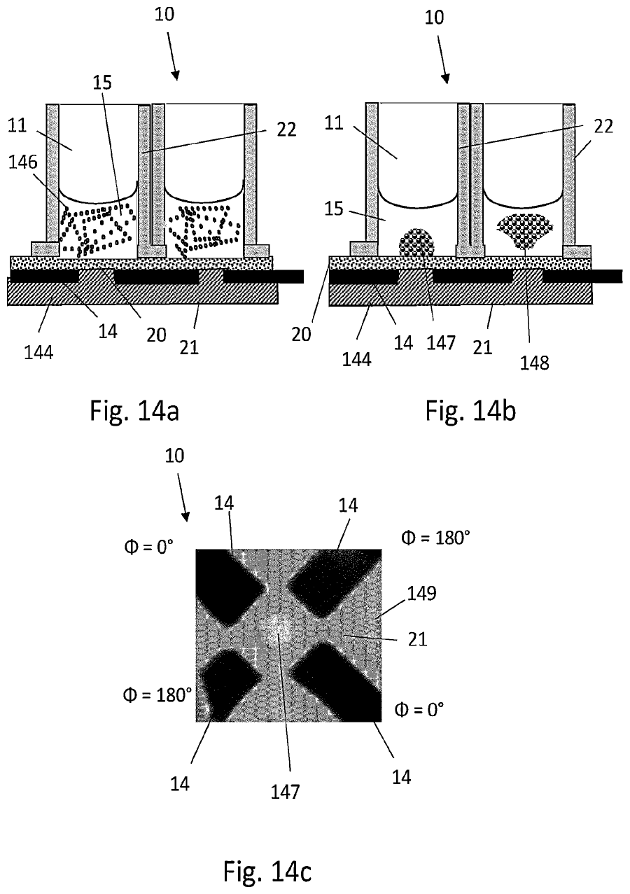

FIGS. 14a-c show a schematic view of the formation of cell aggregates in containers of the device.

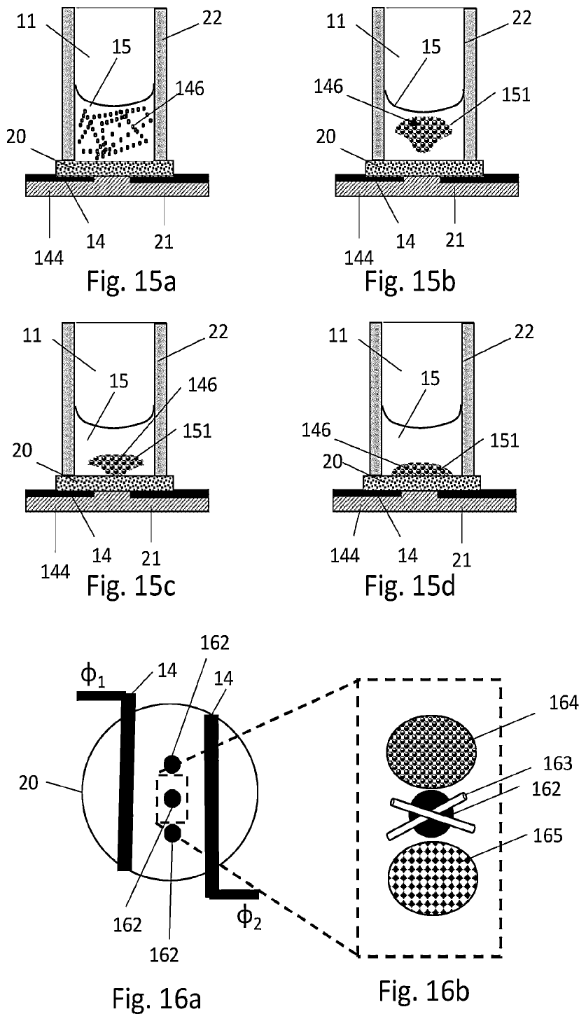

FIGS. 15a-d show a schematic view of different states of manipulation of cells in a container.

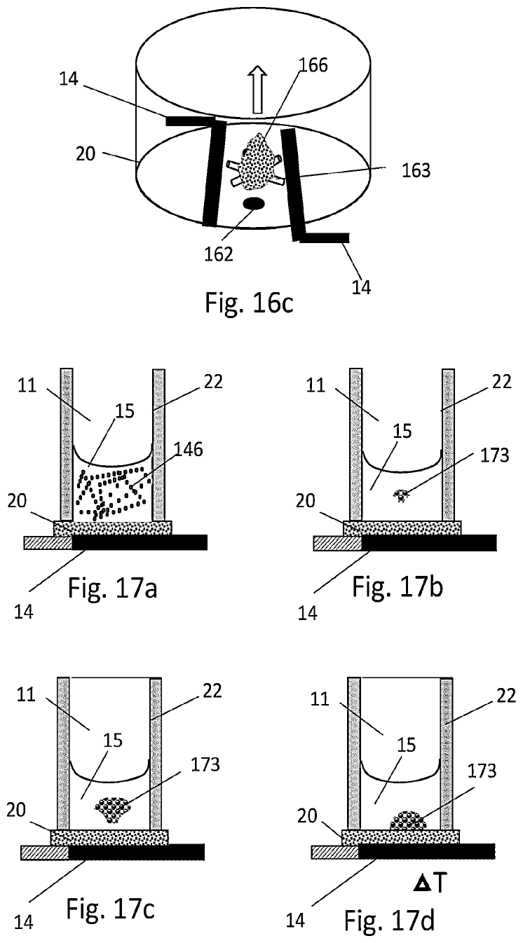

FIGS. 16a-c show a schematic view of a container with island elements for forming and manipulating vascular organoids from the cells.

FIGS. 17a-d show a schematic view of the preparations and the performance of cryopreservation of cell aggregates.

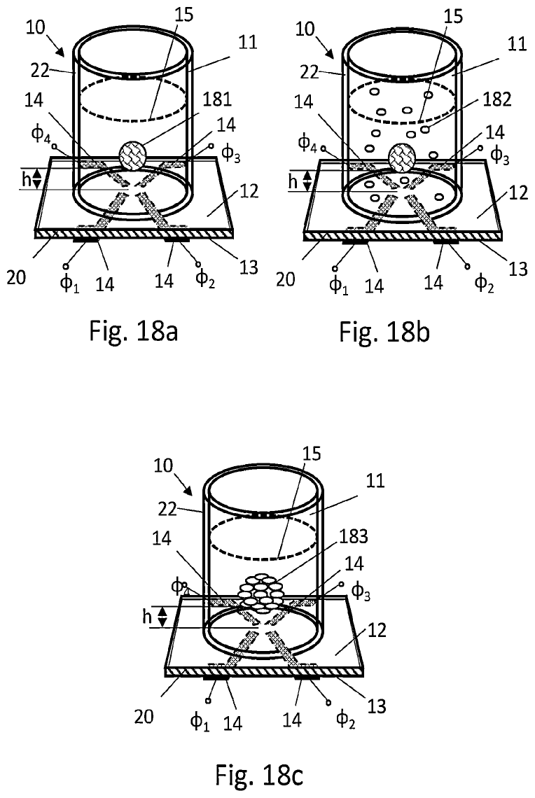

FIGS. 18a-c show a schematic view of the use of capture particles to form organoids from the cells.

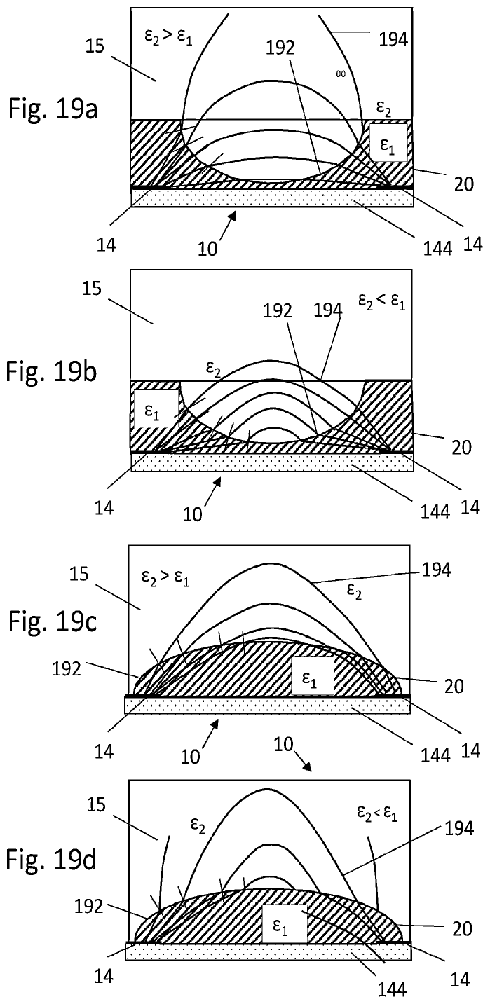

FIGS. 19a-d show a schematic view of examples of separator layers with curved surfaces at the interior space.

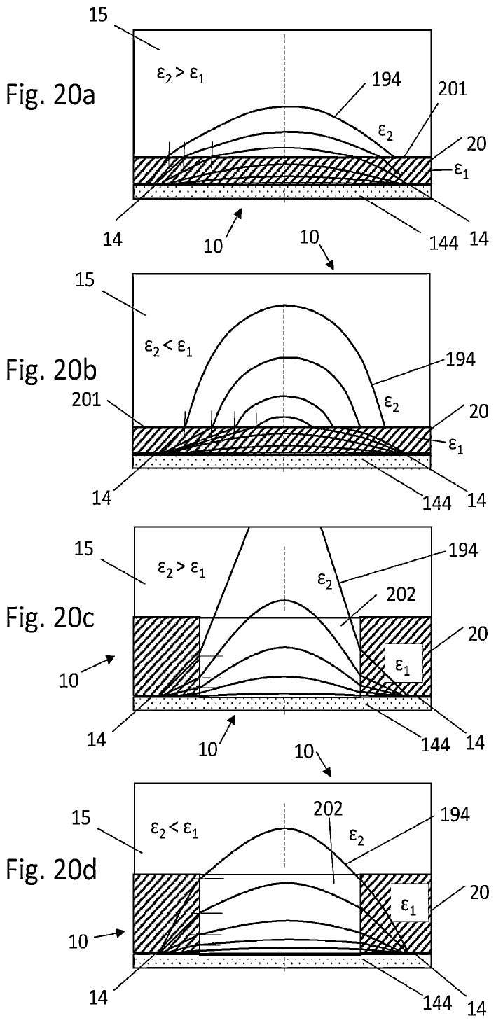

FIGS. 20a-d show a schematic view of example of different ratios of the relative permittivity of the separator layer and the material in the interior space.

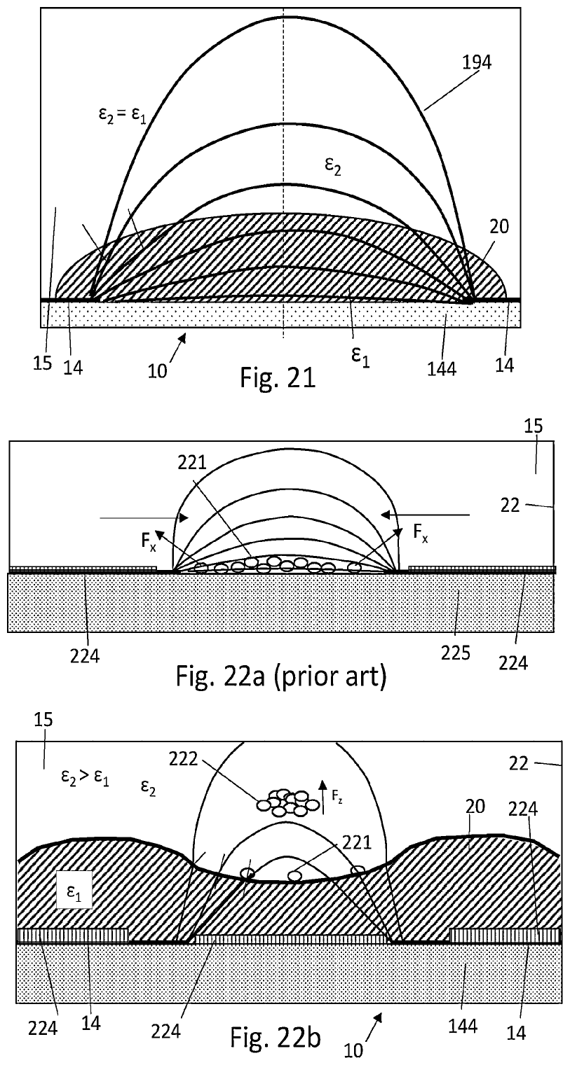

FIG. 21 shows a schematic view of an example with the separator layer and the material in the interior space having the same relative permittivity.

FIGS. 22a, 22b show a schematic view of the levitation effect of the device on cells (b) in comparison to the prior art (a).

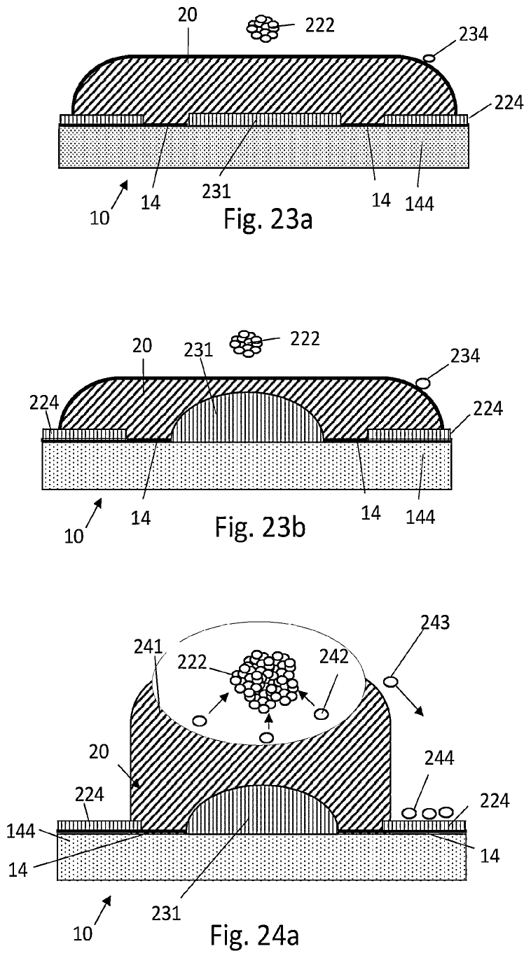

FIGS. 23a, 23b show a schematic view of examples of the separator layer and an isolating element between electrodes.

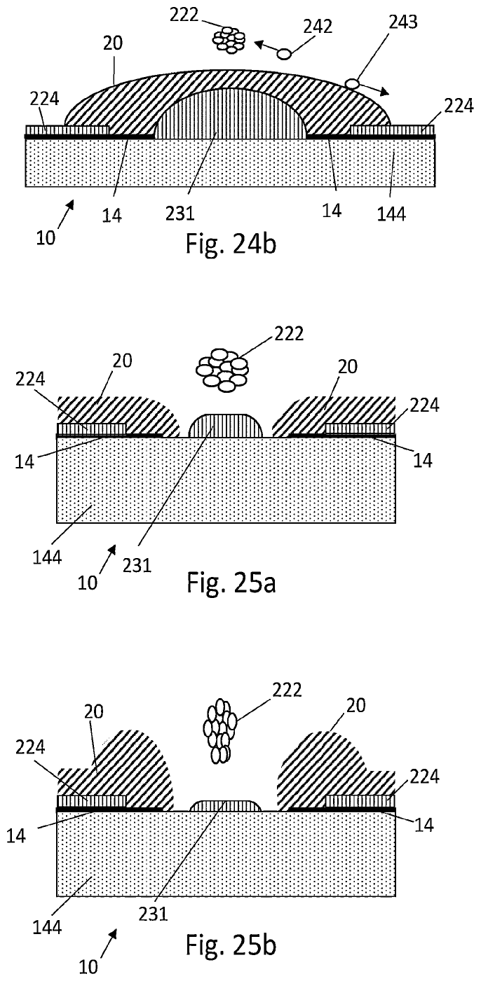

FIGS. 24a, 24b show a schematic view of further examples of the separator layer and an isolating element between electrode.

FIGS. 25a, 25b show a schematic view of examples of the influence of the shape of the separator layer and an isolating element between the electrodes on the cell aggregates.

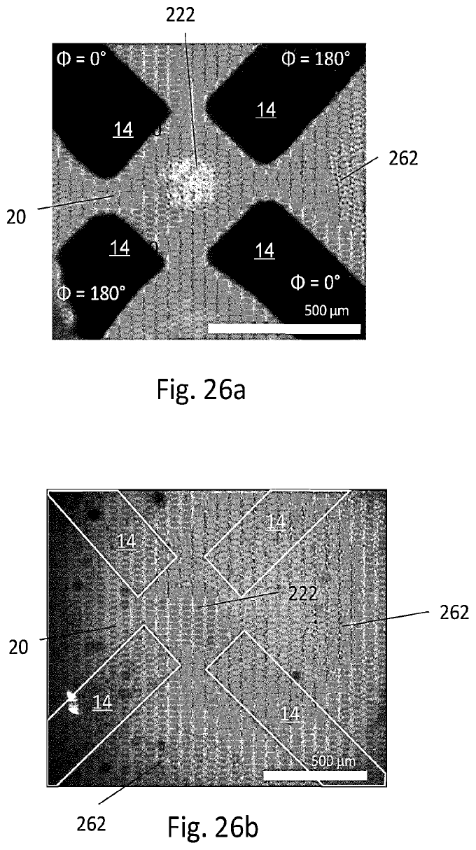

FIGS. 26a, 26b show a schematic view of examples of the effect of four electrodes operated with AC-voltage of a container on the cells.

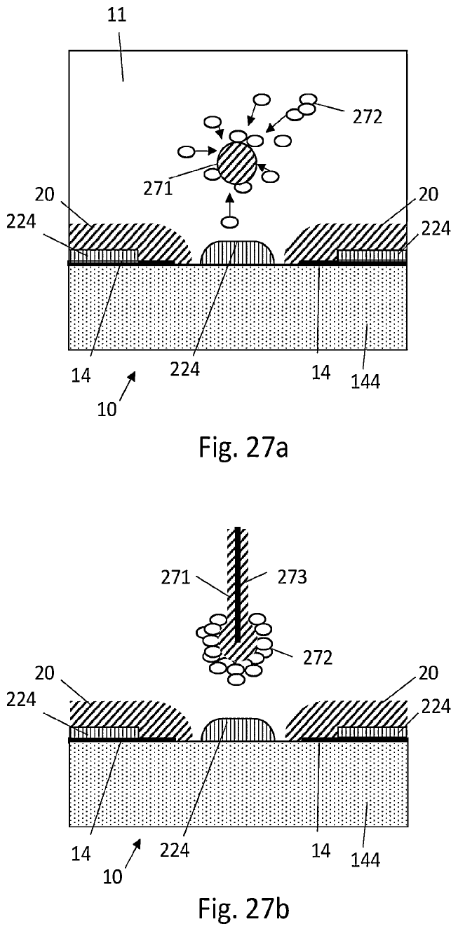

FIGS. 27a, 27b show a schematic view of examples of containers comprising a central catching body.

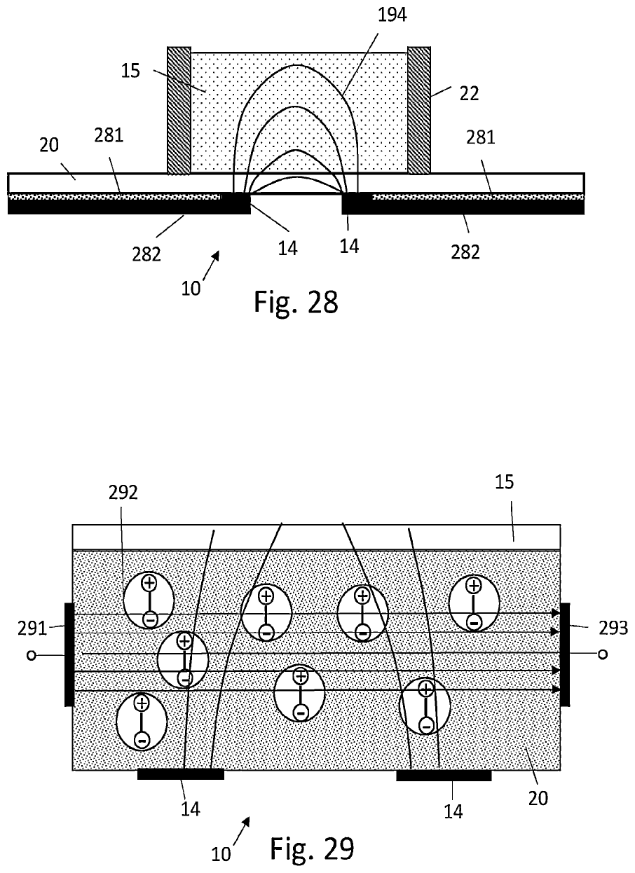

FIG. 28 shows a schematic view of an example of the device having an isolating layer between the electrode and the separator layer.

FIG. 29 shows a schematic view of an example of a dielectric anisotropic switchable separator layer.

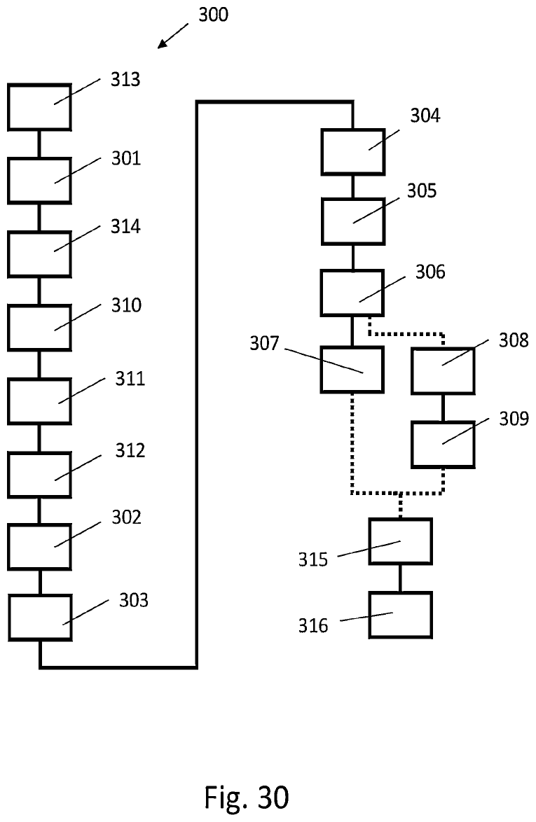

FIG. 30 shows an example of the method for manipulating biological cells.

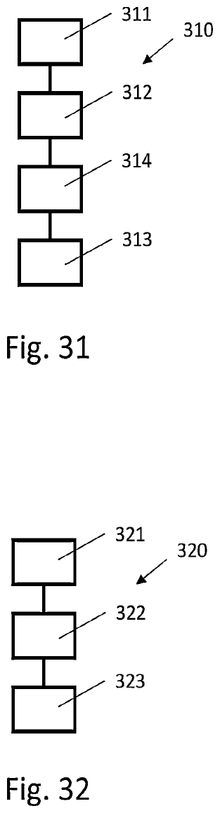

FIG. 31 shows an example of the method of manufacturing a separator layer of the device.

FIG. 32 shows an example of the method of manufacturing a device.

In the following description, reference sign 10 refers to the entirety of the device for manipulating biological cells.

FIG. 1a shows a top view of an example of the device 10. The device 10 of this example, comprises a microwell plate with 96 containers 22 that are designed as wells. The number of containers 22 is exemplary. Therefore, the number of containers 22 may have any number e.g., 6, 12, 24, 48, 384, 1536, or 3456, or any other number.

The containers 22 are attached to a bottom element 21. Each container 22 comprises an interior space 11 that may receive a suspension comprising a nutrient solution 15 and biological cells.

According to FIG. 1b, the bottom element 21 separator layer 20 comprises a top side 12 that faces the interior space 11. Cells may sediment on top side 12. The bottom side 13 of the separator layer 20 faces away from the interior space 11. Furthermore, the bottom side 13 may contact the atmosphere on the outside of the device 10.

The separator layer 20 comprises a high relative permittivity, particularly at the locations of the electrodes 14. The relative permittivity may be in a range from 10 to 10000, preferably from 10 to 1000, further preferably from 20 to 500 for voltage-current waveforms with a frequency in the range from 1 kHz to 10 MHZ. The separator layer 11 may comprise a material having ferromagnetic properties.

The separator layer 20 may comprise particles having a high relative permittivity. Those particles may for example comprise a titanate of an alkaline earth metal, preferably CaTio₃, SrTiO₃, BaTiO₃, Ba₁₋ₓSrₓTiO₃ and/or combinations thereof. The material mixture of the separator layer 20 may comprise those particles in a ratio between 10% to 60% by volume, further preferably between 30% and 50% by volume, most preferably of at most 40% by volume.

The following table 1 shows the relative permittivity of a group of titanates:

TABLE 1
Titanates and their relative permittivity

| | |
|---|---|
| Magnesium titanate | ε = 17-19 |
| Calcium titanate | ε = 200-250 |
| Barium titanate | ε = 300-1000 |
| Strontium titanate | ε = 250-334 |
| BaO-MgO-TiO₂-mixture | ε = 34-1000 at 1 MHz |
| BaO-CaO-TiO₂-mixture | ε = 34-1400 at 1 MHz |

Those particles may be embedded in a polymer of the material mixture. Polymers that are used for bottom elements 21 of containers 22 have a low relative permittivity. Thus, those bottom elements 21 mitigate electric fields that extend through the bottom elements 21.

The particles with a high relative permittivity embedded in the polymer may increase the total relative permittivity of the material mixture. Thus, the material mixture has a lower mitigation of electric fields extending through the bottom element.

In this example, the container 22 comprises four electrodes 14. The electrodes 14 are not located in the interior space 11 of the container 22. Contrary, the electrodes 14 are processed on the bottom side 13 of the separator layer 20. Thus, the separator layer 20 is arranged between the electrodes 14 and the interior space 11.

In another example, the electrodes 14 may be arranged on the top side 12. Then, the separator layer 20 may cover the complete surface portion of the electrodes 14 that abut the interior space 11. Thus, also in this example, the separator layer 20 is arranged between the interior space 11 and the electrode 14 such that the electrode 14 is arranged outside the interior space 11.

In the four-electrode example of FIG. 1b, two electrodes 14 may form a pair. For example, two electrodes 14 on opposite sides of the container 22 may form a pair. An alternating electric potential Φ₁, Φ₂ may be applied to each pair of electrodes 14, such that the pairs of electrodes 14 generate two alternating electric fields. The frequency of the alternating electric fields may be in the radiofrequency spectrum, this being a high frequency electric field which may also be called radiofrequency field. The alternating electric potential of each electrode 14 of a pair of electrodes 14 may have the same phase. The phase difference between Φ₁ and Φ₂ may for example be 180°.

The container 11 may be filled with a cell suspension 15. The dashed ellipse indicates the meniscus of the suspension 15. The suspension 15 fills half of the interior space 11.

The frequency, the field strength, and electrode arrangement may define a mode of the high frequency electric field. Depending on the mode, the resulting electric field polarizes the biological cells in the suspension. The polarization has the effect that the electric field causes forces on the cells. Those forces act on the cells without physical contact.

The forces cause the cells to form aggregates 16, 17, 18. In this example, the cell aggregate 17 levitates at a height h corresponding approximately to the distance between the electrodes 14 in the center of the container 11. The height h may be adjusted between 1 μm and several mm via the field strength.

The cell aggregate 16 may accumulate on the bottom of the container 11 between the electrodes 14. The cell aggregate 18 may be pulled at and on the electrodes, or into the narrowest gaps.

For the contactless, mechanics-free generation of forces on cells, metal electrodes of a width between 10 μm and 1 mm and corresponding feed lines may be processed on the bottom side 13 of the separator layer 20. The processing may for example be performed with semiconductor technology, screen printing, spotting, etc.

The total height of the microwell plate may for example be in the range of 5 mm to 20 mm. The depth of the interior space 11 may for example be in the range of 1 mm to 15 mm. The diameter of the interior space 11 may for example be in the range of 0.5 mm to 10 mm.

FIGS. 2a to 2f show various electrode shapes in a plan view to the bottom side 13. The ring indicates the position of the wall of the container 22, for example a well cylinder, relative to the electrodes 14. The electrodes 14 comprise end portions 26 in the center of the container 22.

FIG. 2a shows a linear shape of the electrodes 14. The electrodes 14 may be operated with more than two phases Φ1, Φ2, Φ3, Φ4 as shown in FIG. 2a. Alternatively, the electrodes 14 may be operated with a pure alternating electric field having two phases Φ1, Φ2 as shown in FIG. 2b. The electrodes 14 shown in FIG. 2b have a cross shape. The electrodes 14 in FIG. 2c have a Y-shape.

According to FIGS. 2d to 2f, the end portions 26 may comprise a plurality of circular, T-shaped and/or triangular projections extending along the bottom element 21. Those projections may shape the electric field generated by the electrodes 14.

The generated patterns of electromagnetic field maxima and minima of high-frequency voltage-current waveforms with a frequency range from 1 kHz to 10 MHz can be predetermined by electrode position, shape and/or surface area.

Depending on the induced polarization of the cells, the choice of the frequency of the electric field and/or the conductivity of the suspension, i.e., cell cultivation medium, results in positive or negative dielectrophoresis, wherein the dielectrophoresis describes the type of force and cell movement acting on the cells via the electric field.

FIG. 3a shows a diagram of cell spectra showing the force as a function of frequency that is shown logarithmically. The diagram shows three curves a), b) and c) which comprise different conductivities of the suspension 15. Curve a) has the suspension 15 with the lowest conductivity, wherein curve c) has the suspension 15 with the highest conductivity.

The polarization of the cells inter alia depends on the conductivity of the suspension and the frequency of the electric field. The polarization in the region of arrow 31 is opposite to the polarization in the region of arrow 32. Thus, in the case of curves a) and b) the polarization of the cells may switch depending on the applied frequency of the electric field.

Furthermore, the polarization of the cells in the region of arrow 31 indicates positive dielectrophoresis. The polarization of the cells in the region of arrow 32 indicates negative dielectrophoresis. Consequently, the applied electric field may set positive or negative dielectrophoresis if the suspension 15 has a suitable conductivity, for example as shown by curve a) or b).

In the kHz-range from 1 kHz to 100 kHz, negative dielectrophoresis occurs without exception. At lower conductivities a) and b), the polarization of the cells changes polarity between 100 kHz and 50 MHZ resulting in positive dielectrophoresis. Above 50 MHZ, negative dielectrophoresis occurs again.

Negative dielectrophoresis can be achieved over the entire frequency range by selecting a high conductivity, as is inherent in common cell cultivation media. Negative dielectrophoresis is preferable for cell manipulation because the cells migrate to areas of low field strength, which also minimizes exposure to the electric fields. High frequencies are also preferable, since the rapid change results in lower deflections of the charge carriers, for example ions. Thus, reduces irreversible processes, further.

The dielectric properties of the bottom element determine a cut-off frequency. Frequencies up to 100 kHz are feasible. At higher frequencies, the coupling of the electric field deteriorates because the relative permittivity of the bottom element decreases with increasing frequency. This is caused by the average relaxation time of the charge carriers/dipoles that is exceeded, i.e., they can no longer follow the high frequent change of the electric field.

If positive dielectrophoresis is set, the cells are pulled in the direction of the electromagnetic field maxima. Those are always located close to the electrodes 14, particularly at the electrode edges and the closest distances between the electrodes 14. The geometric shape of the electrodes 14, for example as shown in FIGS. 2a to 2f, may thus be used to determine the arrangement of the cells as a group or line or even a cluster at high cell density with high precision.

If negative dielectrophoresis is set, the cells are inversely polarized as in the case of positive dielectrophoresis. The cells then move in the direction of the field minima, collect on surfaces under which no electrodes are present, or are lifted.

FIG. 3b shows a spatial representation of the different behavior of the cells and the effect of the induced forces. The electrodes 14 are arranged as a quadrupole and controlled via two phases Φ₁, Φ₂. In positive dielectrophoresis, the cells aggregate at the locations 36 of highest field strength and are drawn to the bottom of the container 22. In the present example, the applied field produces a field minimum at the center 33 of the container 22. The field minimum flares upward in a funnel shape 34.

In the case of negative dielectrophoresis, at low voltage, the cells gather at the bottom at the center 33 of the container 22. If the voltage is increased, the electric force from the electric field on the cells exceeds the sedimentation force acting on the cells. The electric force causes the cell aggregate 17 to levitate. Thus, those electric force may also be called levitation force.

The levitation force causes the cell aggregate 17 to levitate in the force funnel 34 in free suspension at a height where the levitation force is exactly equal to the sedimentation force. Furthermore, the levitation force holds the cell aggregate 17 on the central axis above the electrodes 14, as shown in FIG. 3b.

If the container 22 comprises different cell types with different dielectrophoresis spectra, positive and negative dielectrophoresis may occur at specific frequencies of the alternating electric field. This is one condition for co-cultivation in the device.

FIGS. 4a to 4f show the arrangements of electrode 14 of FIGS. 2a to 2f for cells with negative dielectrophoresis. Cell aggregates 41, 42 may form while negative dielectrophoresis occurs. The cell aggregate 42 is arranged in the center of the bottom of container 22, wherein the cell aggregate 41 is spaced apart from the center of the bottom of the container 22 in spaces between the electrodes 14. Cell aggregates 41 and cell aggregate 42 may comprise different type of cells in view of their dielectrophoresis spectra.

The cell aggregate 42 may be exposed to any alternating electric field with two to four phases.

The cell aggregates 41 are always exposed only to one alternating field with two phases from the neighboring electrodes 14, i.e. the electrodes 14 that abut the space that the cell aggregate 41 occupies.

Cells and cell aggregates 42 in the center can therefore rotate slowly, for example, at 5 revolutions per second to 0.001 revolutions per second. Furthermore, those cell aggregates 42 may also be levitated so that they may be easily removed while the cell aggregates 41 stay on the bottom of the container 22.

Thus, the cells and cell aggregates 41, 42 may be arranged in patterns in those arrays of dry electrodes 14. Those arrays allow a barrier-free co-cultivation of cells. For example, the cell types of cell aggregation 41 may be a nurse culture that conditions the nutrient solution for cell types of cell aggregation 42.

FIGS. 4c and 4f show examples of parallel formation, propagation, and culture of thirteen organoids or other tissue formations in one container 22 as required in biotechnology, medicine, and pharmaceuticals. Thus, those examples allow the generation of highly ordered organoids/tissues/cell clusters in a 96 microwell plate, i.e., 96*13=1,248 organoids in a 96 microwell plate.

FIGS. 5a to 5f also show the arrangements of electrode 14 of FIGS. 2a to 2f for cells with positive dielectrophoresis. During positive dielectrophoresis, the cell aggregates 51 gather above the electrodes 14 at the bottom of the container 22. The alternating electric fields cannot lift off those cell aggregates 51. Thus, it is of secondary importance how many phases are used since only the alternating electric field of the electrode right below those cells applies a force on the cell aggregates 51.

The device 10 may be operated just at the transition point from positive to negative dielectrophoresis that may be around 100 kHz to 1 MHz according to FIG. 3. In that case, the suspension may comprise at least two cells of different types of dielectrophoresis.

Thus, the combination of FIGS. 4a to 4f and 5a to 5f show that one cell type is pulled to the electrodes 14 and the other cell type is pushed away from the electrodes 14.

After a later change of the frequency to a value at which both show negative dielectrophoresis, the cell groups 42, 51 may be untied to for example configure organoids from different cell types. For example, this is of great importance for vascularization.

FIGS. 6a to 6f show another example of electrodes 14. Those examples comprise electrodes 14 for a two-phase operation. One group of electrodes 14 is connected to a first strip electrode 61, the other group of electrodes 14 is connected to a second strip electrode 62. The strip electrodes 61, 62 have different electric phases. The strip electrodes 61, 62 may connect to electrodes of a plurality of containers 22. Thus, the number of lines may be minimized. The structured of electrodes 14 shown in FIGS. 6d, 6e and 6f allow to generate more than 20 electric field minima. Thus, those structures may provide more than 20 cell aggregates/organoids per container 22.

FIG. 7 shows an example of several containers 22 with the electrode configuration of FIG. 6d. The strip electrode 70 is a zero conductor that connects to one group of electrodes 14 of every container 22 shown in this example. The strip electrode 61 operated at phase Φ1 connects to groups of electrodes 14 of the lower row of containers 22. The strip electrode 62 operated at phase Φ2 connects to groups of electrodes 14 of the upper row of containers 22. This example shows a mirror-image combination of the arrays of electrodes 14 across the rows of a device 10, for example a microwell plate. In this 2-phase operation a multilayer feed line is not required.

For the generation of rotating or wandering multiphase electric fields, crossings of the feed lines are required that may be manufactured with multilevel processing. If those levels of electrodes 14 are arranged outside the interior space on the bottom side 13 of the separator layer 20, the manufacture of the electrodes 14 may be simplified.

FIG. 8 shows a further example of several containers 22 with the electrode configuration of FIG. 6f. This example also shows a mirror-image combination of the arrays of electrodes 14 across the rows of a device 10. This example comprises two groups of strip electrodes 61, 62. Every container 22 comprises electrodes 14 which either connect to the first group of strip electrodes 61 with phase Φ1 or to the second group of strip electrodes 62 with phase Φ2. The first group of strip electrodes 61 is connected to a first contact element 81. The second group of strip electrodes 62 is connected to a second contact element 82. The first and second contact element 81, 82 may be connection pads.

FIG. 9 shows a device 10 comprising a microwell plate with 96 wells as containers 22. The electrical connections with the two contact elements 81, 82 show a 2-phase system in all containers 22. The electrodes 14 have the configuration of FIG. 6a.

FIGS. 10a and 10b show a sectional view of a device 10 comprising a microwell plate 103 with containers 22. The contact elements 81, 82 are arranged on the bottom side of the separator layer 20. The device 10 further comprises a receptacle 101 for the microwell plate 103. The receptacle 101 comprises two spring-loaded contact pins 102 that may be electrically connected to a radio frequency generator (not shown). A lid 105 may cover the microwell plate 103.

In FIG. 10a the microwell plate 103 is separated from the receptacle 101.

FIG. 10b shows the reception of the microwell plate 103 in the receptacle 101. The contact pins 102 connect to the contact elements 81, 82 and make contact. Of course, a variety of other detachable and fixed connections are conceivable.

The electrodes 14 according to the invention capacitively couple in the suspension since the electrodes 14 are arranged outside the interior space and thus the nutrient solution. In comparison to prior art electrodes being in direct contact with the nutrient solution, the electrodes 14 according to the invention have a lower ohmic loss when generating the alternating electric fields. The high number of containers 22 multiplies the reduction of the ohmic loss of the device 10. Thus, the invention avoids an enormous ohmic loss factor of the electrodes being directly coupled to the suspension 15 compared to capacitive coupling. A higher current could compensate the ohmic loss factor of the electrodes that directly couple in the suspension 15. However, this would lead to the electrode processes mentioned above.

FIGS. 11a and 11b show a 96 microwell plate 103. FIG. 11a shows the microwell plate 103 in top view, wherein FIG. 11b shows the microwell plate 103 along section A-A.

According to FIG. 11a, the device 10 comprises an energy storage device 111, e.g. a battery, and an electronic circuit 112 for generating alternating electric fields at the electrodes 14. The energy storage device 111 and the electronic circuit 112 may be integrated in to the microwell plate 103. The device 10 may then be operated autonomously and without any peripheral electronic devices. Miniaturized digital electronic components can be used due to the low currents and the use of square-wave voltage waveforms which are optimal for the purpose of inducing dielectrophoresis. Alternatively, the electronic circuits may be housed in the lid 105, e.g., in a very flat design, and electrically connected via plug-socket or spring-loaded contacts on metal surfaces, etc.

FIG. 12 shows examples of the inner structure of separate layer 20. The separate layer 20 comprises a high relative permittivity that allows a coupling of electromagnetic fields with sufficient force effect onto cells in the suspension 15.

All available polymers, resins, plastic materials have unsuitable, i.e. too low relative permittivities εᵣₑₗ between 2 and 4. In comparison to a direct coupling from the electrode into the suspension this result in a reduction of the effective field strength by a factor of 20 to 40.

The force acting on the cells is proportional to the square of the field strength. To reduce a weakening of the field strength compared to direct coupling in the suspension to a factor of 4, the separator layer 20 requires a total relative permittivity of at least 20.

In the case of direct coupling in the suspension, voltages below 10 Vₚₚ (voltage peak to peak) are used. Otherwise, the applied voltage would heat the electrodes. Consequently, the electrodes would heat the suspension such that bubble formation and electrolysis could occur.

Since according to the invention, the electrodes 14 are arranged outside the interior space 11, the separator layer 20 shields the heat from the electrodes 14 from the suspension 15. Thus, the amplitude of the voltage may be increased to up to 40 Vₚₚ, even higher with appropriate thermal insulation of the bottom side 13.

The reduction of the field strength of the electric field being generated by the electrodes 14 is compensated by the 4-fold higher amplitude of the voltage. If the separator layer 20 comprises a relative permittivity of more than 30, about the same force acts on the cells as with conventional direct coupling in the suspension.

The thickness of the separator layer 20 also weakens the electric field and reduces the desired gradients of the electric field. Therefore, the thickness of the separator layer 20 must be much smaller than distance between the electrodes 14.

Having an electrode spacing of 100 μm to a few mm, a thickness of less than 100 μm, preferably 25 μm or less, reduces the mitigation of the electric field.

Such materials and films are not commercially available. They may be manufactured by mixing cyano resins 121 having a relative permittivity of around 10 with particles, preferable crystals, having ferromagnetic properties and a high relative permittivity εᵣₑₗ of about 100 to 1000, such as barium titanate (BaTiO₃) 122 or barium strontium titanate (Ba1−xSrxTiO3) 123.

Mixtures of cyano resins with an amount of more than 40% by volume of crystals lose transparency. Thus, for inverted microscope imaging of the cells in the container 22 the amount of the crystals should stay below 40% by volume.

FIGS. 12a and 12c show classical mixtures by admixing the crystals 122, 123 with high relative permittivity up to an amount of less than 40% by volume, resulting in a total relative permittivity of less than 25.

FIG. 12a shows the admixture of crystals 122 having a uniform size, for example between 200 nm and 1000 nm, preferably 300 nm.

FIG. 12c shows the admixture of small crystals 122 and big crystals 123 having different sizes, for example 300 nm and 700 nm.

FIGS. 12b and 12d show a preferable configuration of the crystals 122, 123 in the cyano resin 121. The columnar configuration of the crystals 122, 123 may increase the relative permittivity of the separator layer 20, further. The separator layer 20 may for example achieve a total relative permittivity of above 40 with an admixture of the crystals 122, 123 of less than 40% by volume.

FIGS. 13a to 13c show how that desired configuration of the crystals 122, 123 may be achieved.

FIG. 13a shows the stochastic distribution of the crystals 123 in the cyano resin 121 after mixing.

According to FIG. 13b, an electric field is applied perpendicularly through the material mixture during solidification of the separator layer 20, e.g., during annealing or UV light irradiation. That electric field preferably is an alternating radio frequency field. At a suitable frequency in the range of kHz to MHz, the electric field causes the dipoles of the crystals 123 to align and to dielectrophoretically form into a chain in the manner shown in FIG. 13b. If the crystals 122, 123 have different sizes, the electric field forms columns of the shape shown in FIG. 13c.

The alignment in the columns may be washed out or may be very precise. This depends on the period between switching on and off the electric field during the solidification. For example, the crystals 122, 123 align very precisely if the field is switched on until complete solidification.

To avoid a fixed pre-polarization that may occur due to the precisely aligned crystals 122, 123, it is preferred to create rather irregularly arranged columns of the crystals 122, 123, i.e., to switch off the electric field before solidification and to allow position changes of the crystals 122, 123 due to local thermal movements.

FIGS. 14a to 14c show another example of the effects of dielectrophoresis in the device 10.

FIG. 14a shows a sectional view of two containers 22 comprising the cells 146 and the nutrient solution 15. The transparent bottom element 21 comprises the separator layer 20 and an insulating layer 144 insulating the electrodes 14 from the outside. The advantage of adding an insulating layer 144 is that the thickness of the insulating layer 144 does not influence the electric field. The insulating layer 144 stabilizes the bottom element 21 even if the separator layer 20 comprises a thickness in the μm range.

FIG. 14b shows the effect of turning on a radiofrequency field if the conditions are such that negative dielectrophoresis occurs. A cell aggregate 147 forms being required to develop an organoid or tissue formation. In the left container 22, the field strength is low so that the cell aggregate 147 rests on the ground.

In the right container 22, the electric field comprises a higher field strength than in the left container 22. The forces resulting from the electric field lift cell aggregate 148 and hold it free-floating in a field funnel. In this position, depending on the control of the electric field generated by the electrodes 14, the cell aggregate 148 may be rotated and deformed.

The cell aggregate 147 may, for example, be created to improve cryopreservation, since cooling and heating occurs primarily through the bottom element 21 of the microwell plate. Both, the electrodes 14 with good thermal conductivity and the thin separator layer 20 allow the immediate temperature change at the organoid or cell aggregate 147.

For changing the temperature in the cell aggregate 148, the temperature of the ambient nutrient solution must change first. Consequently, for cryopreservation, the electric field must be switched off or the frequency must be switched to positive dielectrophoresis to cause the cell aggregate 148 to sink to the bottom element 21.

FIG. 14c shows a microscope view of such a particle formation in the center between four electrodes 14 on the outside of a separator layer with a total relative permittivity of about 15 at alternating electric field excitation with the indicated phase positions at 100 kHz quadrupole excitation. Negative dielectrophoresis occurs. The surface parts of the bottom element 21 around the electrodes 14 are completely particle-free. The cells 149 which are part of the cell aggregate 147 gather between the electrodes 14.

FIGS. 15a to 15d show an example of a manipulation series, representative of a variety of such cell-particle manipulations.

According to FIG. 15a, the container 22 comprises a cell suspension 15. The electric field is switched off.

After turning on the radiofrequency field and using negative dielectrophoresis, a cell aggregate 151 is formed in the solution, as shown in FIG. 15b.

According to FIG. 15c, the reduction of the field strength causes the cell aggregate 151 to sink to the bottom of the container 22.

In the case of adherently growing cells, the cells spread out on the bottom element 21 and grow, as shown in FIG. 15d.

By adding further cells, for example, further cell layer may be deposited on the existing cell layers since the force of the electric field is not sufficient to lift off the adherent cells.

The sequence according to FIGS. 15a to 15d may also be used shortly before the start of cryopreservation of a complete microwell plate.

FIGS. 16a to 16c show schematically how exemplary vascularized organoids may be generated.

FIG. 16a shows a schematic top view of the electrodes 14 being connected to a radiofrequency source for generation of the alternating electric field and conductive island elements 162 that are not connected to the radiofrequency source. The island elements 162 may, for example, be made of the electrode material. The island elements 162 may be arranged on the same side of the separator layer 20 as the electrodes 14.

The island elements 162 may also be non-conductive. However, the following example refers to conductive island elements 162.

According to FIG. 16b, natural or artificial semipermeable tubes 163 may be added to the solution. Then, the electric field may be switched on alternating with a frequency v, wherein the electric field lines bundle above the electrodes 14 and the conductive island elements 162.

If the tubes 163 show positive dielectrophoresis they will collect above the conductive island elements 162 and at the electrodes 14.

When, after the tubes 162 have been collected, cells being added to the solution will show negative dielectrophoresis at the frequency v because the cells in the cell aggregates 164, 165 have different dielectric properties than the tubes 163. The cells will collect in the field minima in between the electrodes 14 and the island elements 162.

After switching off the alternating electric field and when the cells grow, the two cell aggregates 164, 165 migrate and grow into each other, forming an organoid 166 and enveloping the tubes 163 such that the tubes grow in the forming organoid 166. Nutrients can still reach the interior of the organoid via the tubes 163 even in larger organoids 166, which is required to avoid central necrosis in the organoid 166.

According to FIG. 16c, the organoid 166 has grown large enough to assume a three-dimensional shape. The organoid 166 may then be lifted as indicated by the arrow, by turning the alternating electric field on again. The organoid 166 may be transferred or removed easily as the field forces increase with the third power of the radius of this organoid 166.

FIGS. 17a to 17d show the principle of supporting cryopreservation.

According to FIG. 17a cell 146 are shown in suspension 15.

FIG. 17b shows the formation of an initial cell aggregate 173 in the suspension 15. According to FIG. 17c, the size of the cell aggregate 173 increases.

FIG. 17d shows the cooling during cryopreservation. In particular, the electrodes 14 and the separator layer 20 considerably improve the temperature conduction.

FIG. 18 shows an example of designing organoids using artificial or biological capture particles 181. Those capture particles 181 can be designed as lossy dielectrics like the cells. For example, the capture particles 181 may be functionalized Sephadex particles with a diameter in the range from 1 μm to 300 μm, preferably 50 μm. For designing organoids, the capture particle 181 may be trapped in an electric field minimum, when the electric field is switched on with rotating four phases in this example. The rotating electric field causes the capture particle 181 to levitate at height h above the bottom of the container 22.

According to FIG. 18b. cells 182 are added in a low concentration. The rotating electric field polarizes the cells 182 such that the cells 182 moved to the center. In the proximity of the capture particle 181, dipole-dipole interactions via higher order interactions, for example, quadrupole or octupole orders occur between the cells 182 and the capture particle 181. Those interactions cause the cells 182 to meet the capture particle 181. The cells 182 are pressed to the surface of the capture particle 181 such that the cells 182 can adhere well.

Due to the rotation of the electric field, unlike with a resting alternating field, the surface of the capture particle 181 is uniformly covered with cells 182. This is an important goal in such organoid technologies.

The steps shown in FIGS. 18b and 18c may be repeated with further cells such that cell layer upon cell layer may be deposited on the overgrown capture particle 181. If the rotating electric field is changed to an alternating field, for example by applying two phases to the electrodes 14, the cell load on the capture particle 181 becomes inhomogeneous, i.e., the round body created with the rotating electric field then gets cell projections.

The at least one electrode 14 may be used not only for excitation and generation of forces, but also for measurement of the induced cell polarizations. The excitation-echo technique known from ultrasound may be used, i.e., switching off the electric field very quickly after excitation and recording the decay of the induced polarizations in the cells as current-voltage waveforms with the same electrodes 14. This requires a very high amplification with an amplification factor in the range of 100 to over 1000 with a large signal-to-noise ratio.

FIGS. 19a to 19d show a sectional view of an insulating layer 144 that may be a glass substrate. Flat microelectrodes 14 with a thickness of less than 1 μm may be processed on the glass substrate. The processing of the electrodes 14 may be performed for example by etching methods.

On top of the electrodes 14 and the insulating layer 144 is the separator layer 20 having a high first relative permittivity ε₁ and a low conductivity at a thickness of approx. 50 μm. The separator layer 20 is covered with an aqueous cell suspension 15 wherein the cells are not shown. The suspension 15 has a second relative permittivity 82 in the range of about 80. That relative permittivity ε₂ cannot be significantly changed due to cell biological reasons. The electrodes 14 are completely isolated from the suspension 15 by the separator layer 20.

FIGS. 19a to 19d further show the electric field lines 194 for different geometries of the separator layer 20 and as a function of the ratio of the first and second relative permittivity. In FIGS. 19a and 19c, the second relative permittivity ε₂ is bigger than the first relative permittivity ε₁. In FIGS. 19b and 19d the second relative permittivity 82 is smaller than the first relative permittivity ε₁.

In FIGS. 19a to 19d, the separator layer 20 comprises a curved surface portion 192. FIGS. 19a and 19b show concave surface portions 192. FIGS. 19c and 19d show convex surface portions 192. The electric field lines 194 are refracted at the curved surface portions 192 of the separator layer 20 abutting the suspension 15. This may can influence the field line profile and thus the effect on cells in the suspension 15. The change of the electric field lines 194 depends not only on the change of the relative permittivity on the curved surface portion 192 but also on the geometry of the curved surface portion 192 of the separator layer 20. The electric field can be compressed or distorted into the solution. The cell aggregate adapts to the profile of the electric field lines 194 in shape, e.g. by forming a sphere, disk, or spindle. This may be a means to construct organoids.

The cases in FIGS. 19a and 19c correspond exemplarily to a distribution of relative permittivities of ε₁=40 and Φ₂=80, the cases in FIGS. 19b and 19d corresponds to relative permittivities of ε₁=120 and ε₂=80.

To obtain the curved surface portions, the thickness of the separator layer 20 may vary in the range from 0.5 μm to 100 μm. This may lead to a variation of the relative permittivity of the separator layer 20 in the range from 10 to more than 100.

FIGS. 20a to 20d show two further geometrical examples of the surface 201 of the separator layer 20.

FIGS. 20a and 20b show separator layer 20 with a planar surface 201, the separator layer 20 having a thickness in the range between 1 μm and 100 μm.

FIGS. 20c and 20d show a separator layer 20 having an opening 202 between the electrodes 14. The electrodes 14 are completely covered by the separator layer 20. Thus, the electrodes 14 are arranged outside the interior space 11 and separated from the suspension 15. In particular, the design of an opening 202 may result in significant changes in the change of the electric field lines 194. The ratio of the relative permittivities may calibrate the electric field lines 194. Since the suspension 15 should not be influenced, the calibration of the electric field lines 194 may be achieved by changing the mixing ratio of the separator layer material. For example, the calibration can be determined by adding barium titanate to the separator layer material.

FIG. 21 shows a further example of the separator layer 20. In this example, the separator layer 20 has the same relative permittivity as the suspension 14. In this case, the electric field lines 194 run almost undisturbed across the boundary layer between the separator layer 20 and the suspension 15. The advantage of this combination is that the geometry and surface topography of the separator layer 20 does not influence the profile of the electric field lines 194. Furthermore, the separator layer 20 still isolates the electrodes 14 from the interior space 11 and the suspension 15 and the electric field may be coupled into the suspension 15 without attenuation.

For levitation of the cells for the formation of organoids, a certain distance from the surface is preferable since several relevant cells tend to adhere to surfaces so that no 3-dimensional organoid can be formed. Any contact between the cells and a surface, especially with an artificial material, influences the cell physiology and differentiation which should be avoided. Therefore, not only the lateral collection of cells but their elevation via a vertically directed force F-is required. Since cells exhibit negative dielectrophoresis at frequencies used in the range of 1 kHz to 10 MHZ, the cells are pushed out of the electric field and lifted. In an arrangement of four electrodes 14, the cells may be held in a force funnel.

FIG. 22a shows a conventional electrode chamber according to the prior art. The electrodes 224 are in direct contact with the cell suspension 15. Cells 221 resting on the bottom of container 22 experience a strong force in the lateral direction, but the force in the z-direction is small.

FIG. 22b shows an example with a separator layer 20 having a relative permittivity of ε₁. This results in a lateral as well as strong z component of the force acting on the cells 221. Thus, cells 221 lying on the bottom of the container 22 are also lifted and kept free-floating. In comparison to the prior art configuration shown in FIG. 22a, covering the electrodes 14, even with separator layers 20 having a thickness in the range of 10 μm to 100 μm reduces field losses, avoids electrolytic processes on the metal surface, and even allows biological functionalization of the surface of 223

The device 10 can be further improved by covering the electrodes 14 and the interstices in between the electrodes 14 with an insulating material 224 having a low relative permittivity. This reduces leakage currents and capacitive overcoupling.

FIGS. 23a and 23b show examples of this approach. FIG. 23a shows a planar separator layer 20 with rounded edges. The electrodes 14 are directly connected to the separator layer 20 only in small areas. The main part of the electric field lines originates there. The other areas between the electrodes 14 and the separator layer 20 comprise an insulating material 224, e.g. silicon oxide, silicon nitrite, aluminum oxide of a thickness of about 1 μm.

The rounded edges of the separator layer 20 cause cells 234 located there to be pushed away from the central region. This is of interest, for example, when co-culturing with conditioning cells is performed, wherein the conditioning cells shall not be incorporated into the central organoid 222 but only condition the suspension 15 in a cell physiological direction.

FIG. 23b shows an example, with an insulating layer 231 that is thicker than in the example of FIG. 23a. The insulating layer 231 may improve the focusing of the electric field lines since the electric field lines run around the insulating layer 231. Thus, the insulating layer 231 displaces the electric field lines that would extend at the position of the insulation layer 231.

FIGS. 24a and 24b show further embodiments of this type. According to FIG. 24a, the separator layer 20 comprises a significant height and a depression 241 on the upper surface. The separator layer 20 therefor defines an isolated region in the container 22. The organoid 222 levitates in the depression 241. This configuration simplifies the deflection of cells 243 being arranged at the edge of the separator layer 20. Furthermore, the geometry of the separator layer 20 avoids a mixture of the co-cultured cells 244 with the cells 242 that shall add to the organoid 222.

FIG. 24b shows another example with a separator layer 20 having a curved surface. The curved surface may deflect cells 243 that shall not add to the organoid 222.

FIGS. 25a-b show exampled with different geometric designs of the insulator layer 224 and the separator layer 20. The geometric designs of the insulator layer 224, 231 and the separator layer 20 may interact to position and shape the cell aggregate 222.

FIG. 25a shows an example with a thick central insulating layer 231 and a planar separator layer 20 that is interrupted between the electrodes 14. The resulting electric field lines may then cause weak lateral forces on the organoid 222.

FIG. 25b shows an example with a thin central insulating layer 231, wherein the separator layer 20 has bulky end portions facing the central insulating layer 231. This configuration results in electric field lines that cause high lateral forces on the organoid 222. Thus, the organoid 222 is more compressed in lateral direction than the organoid 222 of FIG. 25a.

FIGS. 26a and 26b show two examples of the effect of four planar electrodes 14 on particles/cells. The electrodes 14 are operated with an AC voltage in the upper kHz range. Furthermore, the electrodes 14 are covered with a layer with a high relative permittivity.

According to FIG. 26a, the separator layer 20 comprises an oxyphene film of 22 μm thickness. The relative permittivity of the oxyphene film was raised to a value of about 30 via water-filled pores with a diameter of 400 nm. The frequency of the electric field of this example is 100 kHz with an amplitude of 20 Vₚₚ. The particles have a size of 20 μm and arranged in aqueous electrolyte solution.

The pores pass through the oxyphene film. Therefore, this type of layer is not yet satisfactory because over a longer period of time, electrode-electrolyte processes may be initiated since the pores reduce separation between the electrodes 14 and the suspension 15.

According to FIG. 26b the separator layer 20 has a thickness of 70 μm. The separator layer 20 comprises cured cyano resin CR5 that is mixed with a portion of 30% by volume barium titanate nanoparticles. The separator layer 20 forms a completely insulating covering of the electrodes 14 and comprises a relative permittivity of about 40. The frequency of the electric field in this example is at 400 KHz with an amplitude of 19 Vₚₚ. The particles have a size of 20 μm and are in aqueous electrolyte solution 15.

In FIG. 26a, the film is transparent enough to see the electrodes 14. In FIG. 26b the location of the four electrodes 14 is indicated by white slanted rectangles oriented towards the middle of the image. In both cases, large cell aggregates 222 are formed, which float three-dimensionally in the form of a sphere in the central region.

The number of trapped cells can be adjusted from a single cell to several thousand via the initial concentration of the cell suspension. To produce heterogeneous organoids, further cells must be added later. Those cells are pulled onto the existing organoids and brought into direct surface contact there via dipole-dipole and quadrupole-quadrupole interactions so that they can form molecular bonds.

In this way, complex cell groups, cell clusters up to organoids with hundreds and thousands of cells can be generated and kept free-floating in solution. Cells 262 that are not in the central region are pushed away from the electrodes 14. Those cells 262 can interact with the organoid cells 222 via the fluid of the suspension 15 but cannot reach them. This is important for co-cultivation, as well as when certain cell type ratios shall remain constant in an organoid.

FIGS. 27a and 27b show the structure shown in FIG. 25b.

According to FIG. 27a, a body 271 of a diameter ranging from a few micrometers to 100 μm is added to the suspension. The material of body 271 may be a mixture of cyano resin and barium titanate. Thus, the body 271 comprises a low conductivity and a strong polarizability in the electric field. Furthermore, the electric field keeps the body 271 very firmly suspended.

In this polarized state the body 271 acts on the much smaller cells 272 via dipole-dipole interactions of the induced polarization charges on all objects like a central capture body. From a wide range around the body 271 the cells 272 move towards the body 271 which is indicated by the arrows. The cells 272 cannot leave the surface of the body 271.

Concerning organoids, the geometry of the capture body 271 can be adapted to the desired shape of the cell formation, e.g., formation as star, ellipsoid, cylinder. The material of the body 271 can also be designed with pores or channels so that nutrient solution can flow through the structure of the body 271. This allows for larger organoids and subsequent vascularization. Thus, the body enhances the collection of cells, especially small cells in the μm range, and the formation of organoids.

To measure the growth and size of an organoid without microscopic observation and non-destructively, in one alternative, the electric field generated via the planar electrodes 14 can be used once and applied with measurement signals.

In another alternative, as shown in FIG. 27b, the capture body 271 can be attached to a microelectrode 273 and guided into the electric field region from above. The microelectrode 273 may be formed as a bead. The material of body 271 has a comparatively high relative permittivity combined with low conductivity and covers of the metal surface of the microelectrode 273. If the device 10 comprises a plurality of containers 22, those electrode bead systems can be introduced into each container 22 from the lid 105 and easily removed when the lid 105 is opened.

Instead of the microelectrode 273, an optical fiber may be inserted alone or in addition to the microelectrode 273 to optically measure the contents of the container 22. In the case of the electrode 273, the measurement signal is an impedance signal. In the case of optical detection, for example, scattered light may be detected.

FIG. 28 shows another example of the device 10. The electrodes 14 are arranged very close to each other. To avoid feed losses via stray capacitances, sections 282 of each electrode 14 is covered with an insulating layer 281. The insulating layer 281 is arranged between the electrodes 14 and the separator layer 20. Thus, the insulating layer 281 isolates the sections 282 from the separator layer 20.

FIG. 29 shows an example to provide dielectrically anisotropic layers from mixtures of polymers and titanates of an alkaline earth metal as separator layer 20, wherein the dielectrically anisotropic layer is switchable. The electrodes 291 generate a transverse electric field in the material of the separator layer 20. This transverse electric field hinders the movement and the displacement of the dipoles 292 of the titanate particles in during a polarization change in the radio frequency field that is generated by the electrodes 14. As a result, the displacement current through the separator layer 20 can be influenced, which leads to a change in the radio frequency field in the suspension 15 in the interior space 11.

In another example, Peltier elements may cool the electrodes or the bottom element on comprising the electrodes.

Furthermore, in another example, a current measurement may be performed to record the pH-value and the size of the organoid.

In another example, dielectrically different beads/microbodies may be arranged at different heights or used with different frequencies. The beads/microbodies may be afflicted with cells of different types. Furthermore, those beads/microbodies may manipulate the cells in the field, e.g., keeping the cells suspended separately and unifying them into an aggregate by changing the alternating electric field.

In a further example, electrodes may be formed by doping mixtures of polymers and titanates of an alkaline earth metal. The doping may create conductive pathways in the mixtures. Those mixtures may for example be barium titanate and strontium titanate plates.

Furthermore, in an example, the radiofrequency field may induce radiofrequency membrane potential changes in the cells. This may influence the uptake and/or the delivery of substances into the cells, influence the differentiation of the cells, synchronize cardiomyocytes or neuronal cells, etc. or influence the mechanotransduction of the cells.

The operating temperature of the device 10 may for example be in the range of −5° C. to 100° C., preferably in the range of 20° C. to 37° C. Furthermore, the pH values of the suspension may for example be in the range of 6 to 8.

The device described above may be used for manipulating biological cells.

Furthermore, the varying electric field coupled capacitively into and externally from a container comprising a suspension with at least one biological cell may be used for manipulating said biological cell in the suspension.

FIG. 30 shows a flowchart representing a method 300 for manipulating biological cells by a device according to the above description. The device may comprise at least two electrodes, wherein the electrical signal is configured such that the at least two electrodes generate an electric field with a stable temporal pattern of minima and maxima within the container.

The method 300 may comprise a first optional step 313. In step 313, the suspension is selected according to its desired conductivity and a frequency of the variable electrical signal is selected to generate a positive or negative dielectrophoresis with respect to the at least one biological cell. Thus, in preparation to the manipulation of the cells, the conditions of the suspension may be set to optimize and simplify the later manipulation process. Furthermore, the frequency of the alternating electrical signal may be chosen such that the dielectrophoretic force suits the requirements of the cell manipulation. The frequency may for example be chosen according to a known profile of the polarization of the cell in dependence on the frequency of the electric field as shown in FIG. 3a.

In a step 301 of the method 300, the suspension comprising at least one biological cell is introduced into the at least one container of the device. If the device comprises a plurality of containers, for example a microwell plate with 1536 wells as containers, each container may receive the suspension. Furthermore, the suspension may be different in each container, e.g., the number or type of cells may differ between each container or the mixture of the nutrient solution of the suspension may differ between each container.

The biological cells may be stem cells, preferably induced pluripotent stem cells.

In a further optional step 314 of the method 300, the at least one cell may be lifted and moved in the container by negative dielectrophoresis. Thus, in step 313, the suspension and the frequency have been chosen such that negative dielectrophoresis occurs with the cells to be manipulated.

In a further optional step 310, the suspension may be conditioned by at least one group of cells. That group of cells may influence the nutrient solution of the suspension such that it becomes optimal for the growth to the type of the cells of the group or another type of cells.

The method 300 may comprise a further optional step 311, after the optional step 310. In optional step 311, the electric field in the container may be used to separate and aggregate the at least one group of cells. This may create a space in the suspension for further cells that may be introduced into the suspension. Those further cells may grow and form an organoid.

Such further cells may be introduced into the suspension in a further optional step 312 of the method 300. The at least one further cell may be moved to a minimum of the electric field and held there for a predefined period to form an organoid and to provide time for cell growth.

In a step 302 of method 300, an electrical signal is applied to the at least one electrode of the device to generate a variable electric field. The variable electric field may also be called an alternating electric field. The variable electric field may have the frequency being chosen in optional step 513.

The electrical signal may have a voltage peak-to-peak value in a range from more than 4 V to 100 V, preferably from more than 10 V to 100 V, more preferably from more than 10 V to 50 V. Furthermore, the at least one electrical signal may be fed in a continuous manner or in non-continuous manner, preferably a pulsed manner.

In a step 303, the variable electric field may be used to move the at least one biological cell to a predefined position in the container. This is a manipulation of the at least one biological cell. For example, the cell may be arranged on the bottom of the container.

The electric field may exert a force in the range of 1 pN to 1000 pN on a cell inside the container at a distance between 10 μm and 5 mm. The electric field may polarize the at least one cell is polarized.

In another optional step 304, the at least one cell may be moved into at least a minimum of the electric field.

In an optional step 305, the suspension may comprise at least two cells. The electric field may form at least two cells in the suspension into at least one cell aggregate.

According to an optional step 306 of method 300, the field strength of the electric field may be reduced to sediment the at least one cell aggregate on a bottom of the container.

In a further optional step 307, the electrical signal is switched off as soon as the at least one cell aggregate has been sedimented.

Alternatively, the method 300 may comprise the optional step 308. In step 308, the electric field exerts a collecting force on the at least one sedimented cell aggregate. This avoids that the cells of the cell aggregate move laterally, such that a flattening of the cell aggregate is avoided.

Furthermore, in optional step 309, the electrical signal is applied permanently or in alternating sequence with a short time application of the electric field.

In an optional step 315 of the method 300, cryopreservation of the suspension may be performed by putting a coolant into direct contact to the at least one electrode. Preferably, the cells have been arranged on the bottom of the container. Since the electrode usually comprises a metal material and the separator layer is very thin, cooling the electrode may efficiently cool the suspension and the cells therein.

The method 300 may further comprise optional step 316. In step 316, the frozen suspension of step 315 or another frozen suspension may be thawed. The thawing may be initiated by feeding at least one electrical signal into the at least one electrode at a frequency in a range of 1 kHz to 10 MHz to heat the electrode. As described in analogy in step 315, the metal material of the electrode and the thin separator layer may efficiently transfer the heat to the suspension and the cells.

The at least one cell may be observed microscopically in the container and a behavior, and an increase of the cell may be recorded.

Furthermore, the at least one cell may be measured electrically and/or dielectrically by an induced polarization by the electric field.

The steps of method 300 mentioned above may be performed in any order that is sensible and logical.

FIG. 31 shows a flowchart of a method 310 of manufacturing a separator layer of the device described above.

In a first step 311 of method 310 at least one liquid polymer having a first relative permittivity is provided. The liquid polymer may be a cyano resin from the group: cyanoethyl pullulan (CRS), cyanoethyl poly (vinyl alcohol) (CRV), cyano resin type M (CRM). Furthermore, the first relative permittivity may be above 10.

In second step 312, the plurality of particles, preferably microcrystals, having a second relative permittivity are mixed with the at least one liquid polymer to obtain a separator layer mixture. Those particles may for example comprise a titanate of an alkaline earth metal of the group: CaTio₃, SrTiO₃, BaTiO₃, Ba₁₋ₓSrₓTiO₃ and/or combinations thereof.

The material mixture of the separator layer may comprise those particles in a ratio between 10% to 60% by volume, further preferably between 30% and 50% by volume, most preferably of at most 40% by volume.

The particles may have a uniform size or may have at least two different sizes.

In an optional step 314 of method 310, the separator material mixture may be applied on the at least one electrode in a closed layer preferably by sputtering, spin coating, screen printing and/or depositing in a sol-gel process.

Furthermore, the method 310 further comprises step 313. In step 313, the separator material mixture is solidified to obtain a separator layer.

An electric field may be applied to the separator material mixture during at least a fraction of the solidification process for alignment, collection, and chain formation of the particles in the liquid polymer. This may produce ordered, columnarly aligned particle regions and/or particle clusters in the separator layer.

Furthermore, the separator material mixture may be solidified to form a separator layer, wherein the electric field is applied perpendicularly to the separator material mixture during its solidification.

FIG. 32 shows a flow chart of method 320 of manufacturing a device described above.

In a first step 321, the container for cultivating biological cells is provided. Furthermore, a plurality of containers may be provided, e.g. as well of a microwell plate.

In a second step 322 layer of the separator layer may be provided. The layer may be provided according to method 310 described above.

In a third step 323, the at least one electrode for forming and manipulating the biological cells is provided. The separator layer separates the electrode from the interior space of the container.

Some embodiments of the invention mentioned above refer to method type claims whereas other embodiments refer to the device type claims. Unless otherwise notified, a person skilled in the art will realize that in addition to any combination of features belonging to one type, the application also discloses any combination between features relating to different types. However, all features can be combined providing synergetic effects that are more than the simple summation of the features.

Although the drawings and the foregoing description illustrate and describe the invention in detail, the drawings and the foregoing description only provide examples which do not restrict the invention. The disclosed embodiments do not limit the invention. A skilled person practicing the claimed invention, from a study of the drawings, the disclosure, and the dependent claims can understand and effect other variations to the disclosed embodiments.

Any reference signs in the claims should not be construed as limiting the scope of the present invention to the exemplary elements used in the drawings.

1. Device for manipulating biological cells, the device (10) comprising:
at least one container (22) for cultivating biological cells, the container (22) having an interior space (11), and
at least one electrode (14) for manipulating the biological cells,
characterized in that the device (10) comprises at least one separator layer (20), the at least one separator layer (20) being arranged at least between the at least one electrode (14) and the interior space (11) such that the at least one electrode (14) is arranged outside of the interior space (11).

2. Device according to claim 1, characterized in that at least one bottom element (21) of the at least one container (22) comprises the separator layer (20) and the at least one electrode (14) is arranged outside the interior space (11) at the bottom element (21).

3. Device according to claim 2, characterized in that the at least one bottom element (21) comprises a thickness of at most 200 μm, preferably of at most 100 μm, further preferably of at most 25 μm, at least at the at least one electrode (14).

4. Device according to claim 2 or 3, characterized in that the at least one electrode (14) is arranged between the bottom element (21) and an electrically insulating layer (144).

5. Device according to claim 1, characterized in that the at least one electrode (14) is arranged at least partially in the at least one container (22) and, at least inside the container (22), is coated with the separator layer (20).

6. Device according to any one of claims 1 to 5, characterized in that the separator layer (20) comprises materials having a relative permittivity in a range from 10 to 10000, preferably from 10 to 1000, further preferably from 20 to 500 for voltage-current waveforms with a frequency in the range from 1 kHz to 10 MHz and preferably comprises ferromagnetic properties.

7. Device according to any one of claims 1 to 6, characterized in that the separator layer (20) comprises particles (122, 123) having a crystal structure, preferably micro-crystals, the particles (122, 123) having a uniform size in the range from 100 nm to 1000 nm, preferably 300 nm, or the particles (122, 123) having different sizes in the range from 100 nm to 1000 nm, preferably 300 nm and 700 nm.

8. Device according to claim 7, characterized in that the particles (122, 123) are arranged in a columnar manner in the separator layer (20) between two opposite sides of the separator layer (20).

9. Device according to any one of claims 1 to 8, characterized in that the separator layer (20) comprises a titanate of an alkaline earth metal, preferably CaTio₃, SrTiO₃, BaTiO₃, Ba₁₋ₓSrₓTiO₃ and/or combinations thereof, preferably in a ratio between 10% to 60% by volume, further preferably between 30% and 50% by volume, most preferably of at most 40% by volume.

10. Device according to any one of claims 1 to 9, characterized in that the separator layer (20) comprises at least one polymer (121), preferably a cyano resin, more preferably CRS, CRV and/or CRM, further preferably with a relative permittivity above 10.

11. Device according to any one of claims 1 to 10, characterized in that the separator layer (20) has a total relative permittivity in a range of from 10 to 200, preferably from 16 to 120, more preferably from 20 to 120, for voltage-current waveforms having a frequency in the range of from 1 kHz to 10 MHZ.

12. Device according to any one of claims 1 to 11, characterized in that the separator layer (20) comprises at least one curved surface region (192) at the interior space (11).

13. Device according to claim 12, characterized in that the at least one curved surface region (192) comprises at least one convex and/or at least one concave portion.

14. Device according to any one of claims 1 to 13, characterized in that the separator layer (20) comprises at least two regions (224, 225), the at least two regions (224, 225) having a different relative permittivity, wherein the at least two regions (224, 225) preferably comprise different materials and/or different material mixture ratios.

15. Device according to any one of claims 1 to 14, characterized in that the at least one electrode (14) comprises a base metal, in particular aluminum or nickel; an alloy of base metals; or at least one plotter-writable conductive ink or paste.

16. Device according to any of claims 1 to 15, characterized in that the device (10) comprises at least two electrodes (14) or comprises at least four electrodes (14) in a quadrupole arrangement or comprises at least eight electrodes (14) in an octupole arrangement.

17. Device according to one of claim 15 or 16, characterized in that the electrodes (14) are electrically connected to different phases of a multiphase voltage source or are electrically connected in pairs to one phase of a multiphase voltage source.

18. Device according to any one of claims 1 to 17, characterized in that the at least one electrode (14) has at least one section, which is cross-shaped or Y-shaped.

19. Device according to any one of claims 1 to 18, characterized in that the at least one electrode (14) comprises an end piece having a circular, triangular, square, or T-shaped cross-sectional area.

20. Device according to any one of claims 1 to 19, characterized in that the at least one electrode (14) is linear or zigzag-shaped and/or has a plurality of preferably triangular projections extending along the bottom element (21).

21. Device according to any one of claims 1 to 20, characterized in that the device (10) comprises a plurality of containers (22) and a plurality of electrodes (14), wherein at least one of the plurality of electrodes (14) is arranged on each container (22), wherein the device (10) preferably is formed as a microwell plate.

22. Device according to claim 21, characterized in that a first group of the plurality of electrodes (14) is electrically connected to a first phase of a multiphase voltage source via a first electric line (71) and a second group of the plurality of electrodes (14) is electrically connected to a second phase of the multiphase voltage source via a second electric line (72), wherein at least one electrode (14) from each group is disposed on each container (22).

23. Device according to any one of claims 1 to 22, characterized in that the device (10) further comprises at least one energy storage device (111) and at least one electronic circuit (112) for generating voltages having a frequency at least in the range between 1 kHz and 10 MHz, the at least one electronic circuit (112) electrically connecting the at least one electrode (14) to the at least one energy storage device (111).

24. evice according to claim 23, characterized in that the at least one electrode (14) is arranged on a first module (106) and the at least one electronic circuit (112) is arranged on a second module (105) being detachable from the first module (106), the at least one electrode (14) being electrically connected to the at least one electronic circuit (112) via a detachable electrical contact that is arranged between the first module (106) and the second module (105).

25. Device according to any one of claims 1 to 24, characterized in that the device (10) comprises at least one conductive or insulating island element (162) arranged electrically separated from the at least one electrode (14) on the container (22), wherein the separator layer (20) separates the conductive or insulating island element (162) from the interior space (11).

26. Device according to claim 25, characterized in that the at least one conductive or insulating island element (162) has a length in the range of 1 μm to 200 μm.

27. Device according to one of claims 1 to 26, characterized in that the interior space (11) comprises at least one bead, tube (163) and/or wire.

28. Device according to one of claims 1 to 27, characterized in that at least a portion of the device (10) comprises a functional material surface.

29. Device according to any one of claims 1 to 28, characterized in that the biological cells are stem cells, preferably induced pluripotent stem cells.

30. Method for manipulating biological cells by a device according to any one of the preceding claims, wherein the method (300) comprises the following steps:
Introducing (301) a suspension comprising at least one biological cell into the at least one container;
Applying (302) an electrical signal to the at least one electrode to generate a variable electric field;
Moving (303) the at least one biological cell to a predefined position in the container by the variable electric field, thereby manipulating the at least one biological cell.

31. Method according to claim 30, characterized in that the electrical signal has a voltage peak-to-peak value in a range from more than 4 V to 100 V, preferably from more than 10 V to 100 V, more preferably from more than 10 V to 50 V.

32. Method according to claim 30 or 31, characterized in that the at least one electrical signal is fed in a continuous manner or in non-continuous manner, preferably a pulsed manner.

33. Method according to any one of claims 30 to 32, characterized in that the device comprises at least two electrodes, wherein the electrical signal is configured such that the at least two electrodes generate an electric field with a stable temporal pattern of minima and maxima within the container.

34. Method according to claim 33, characterized in that the electric field exerts a force in the range of 1 pN to 1000 pN on a cell inside the container at a distance between 10 μm and 5 mm.

35. Method according to claim 30 or 34, characterized in that the at least one cell is moved (304) into at least a minimum of the electric field.

36. Method according to any one of claims 30 to 35, characterized in that the at least one cell is polarized by the electric field.

37. Method according to claim 36, characterized in that the suspension comprises at least two cells, wherein the cells in the suspension are formed (305) into at least one cell aggregate by the electric field.

38. Method according to claim 37, characterized in that the at least one cell aggregate is sedimented (306) on a bottom of the container by a reduction of a field strength of the electric field.

39. Method according to claim 38, characterized in that the electrical signal is switched off (307) as soon as the at least one cell aggregate has been sedimented.

40. Method according to claim 38, characterized in that a collecting force is exerted (308) on the at least one sedimented cell aggregate by the electric field.

41. Method according to claim 40, characterized in that the electrical signal is applied (309) permanently or in alternating sequence with a short time application of the electric field.

42. Method according to any one of claims 30 to 41, characterized in that after the step of introducing (301) the suspension, the suspension is conditioned (310) by at least one group of cells.

43. Method according to claim 42, characterized in that after conditioning (310) the suspension, the at least one group of cells is separated and aggregated (311) by the electric field in the container.

44. Method according to claim 43, characterized in that at least one further cell is introduced (312) into the suspension, wherein the at least one further cell is moved to a minimum of the electric field and held there for a predefined period.

45. Method according to any one of claims 30 to 44, characterized in that the at least one cell is observed microscopically in the container and a behavior, and an increase of the cell is recorded.

46. Method according to any one of claims 30 to 45, characterized in that the method further comprises the following step before the step of introducing (301) the suspension:
Generating (313) positive or negative dielectrophoresis with respect to the at least one biological cell by selecting the suspension according to its desired conductivity and selecting a frequency of the variable electrical signal.

47. Method according to claim 46, characterized in that the at least one cell is lifted (314) and moved in the container by negative dielectrophoresis.

48. Method according to any one of claims 30 to 47, characterized in that, after the step of applying (302) the electrical signal to the at least one electrode, the method further comprises at least one of the following steps:
Cooling (315) the at least one electrode by direct contact with a coolant while performing cryopreservation of the suspension; and/or
Heating (316) the at least one electrode by feeding at least one electrical signal into the at least one electrode at a frequency in a range of 1 kHz to 10 MHZ when thawing a frozen suspension.

49. Method according to any one of claims 30 to 48, characterized in that at least one cell is measured electrically and/or dielectrically by an induced polarization by the electric field.

50. Method according to any one of claims 30 to 49, characterized in that the biological cells are stem cells, preferably induced pluripotent stem cells.

51. Method of manufacturing a separator layer of the device according to any one of claims 1 to 29, the method (310) comprising the following steps:
Providing (311) at least one liquid polymer having a first relative permittivity;
Mixing (312) a plurality of particles, preferably microcrystals, having a second relative permittivity with the at least one liquid polymer to obtain a separator layer mixture;
Solidifying (313) the separator material mixture to obtain a separator layer.

52. Method according to claim 51, characterized in that an electric field is applied to the separator material mixture during at least a fraction of the solidification process thereof for alignment, collection, and chain formation of the particles in the liquid polymer to produce ordered, columnarly aligned particle regions and/or particle clusters in the separator layer.

53. Method according to claim 51 or 52, characterized in that in the step of mixing (312) of the plurality of particles with the at least one liquid polymer, the particles have a uniform size and preferably a volume ratio of at most 40% with respect to the liquid polymer.

54. Method according to one of claim 51 or 53, characterized in that in the step of mixing (312) of the plurality of particles with the at least one liquid polymer, the particles have at least two different sizes.

55. Method according to any one of claims 51 to 54, characterized in that the separator material mixture is solidified to form a separator layer, wherein the electric field is applied perpendicularly to the separator material mixture during its solidification.

56. Method according to any one of claims 51 to 55, characterized in that, before solidifying (313) the separator material mixture, the separator material mixture is applied (314) on the at least one electrode in a closed layer preferably by sputtering, spin coating, screen printing and/or depositing in a sol-gel process.

57. Method of manufacturing a device according to any one of claims 1 to 29, the method (320) comprising:
providing (321) the container for cultivating biological cells,
providing (322) a layer of the separator layer,
providing (323) the at least one electrode for forming and manipulating the biological cells, wherein the electrode is separated from the interior space of the container by the separator layer.

58. Method according to claim 57, wherein the separator layer is manufactured according to the method of any one of claims 51 to 56.

59. Use of the device of any one of the claims 1 to 29 for manipulating biological cells.

60. Use of a varying electric field coupled capacitively into and externally from a container comprising a suspension with at least one biological cell for manipulating said biological cell in the suspension.
